(19) United States
(12) Patent Application Publication
(10) Pub. No.: US 2017/0334977 A1
(43) Pub. Date: Nov. 23, 2017

(54) Targeting Apolipoprotein E (APOE) in Neurologic Disease

(71) Applicant: The Brigham and Women's Hospital, Inc., Boston, MA (US)

(72) Inventors: Oleg Butovsky, Boston, MA (US); Susanne Krasemann, Hamburg (DE); Charlotte Madore, Brookline, MA (US); Howard Weiner, Brookline, MA (US)

(21) Appl. No.: 15/520,039

(22) PCT Filed: Oct. 20, 2015
(86) PCT No.: PCT/US15/56492

§ 371 Date: Apr. 18, 2017

Related U.S. Application Data
(60) Provisional application No. 62/065,876, filed on Oct. 20, 2014.
(60) Provisional application No. 62/080,628, filed on Nov. 17, 2014.

(51) Int. Cl. C07K 16/18; C12N 15/113

(52) U.S. Cl. CPC C07K 16/18; C12N 15/113; C07K 2317/76

(57) ABSTRACT

Methods for treating neurologic diseases, e.g., amyotrophic lateral sclerosis (ALS) and multiple sclerosis, by modulating the APOE-TGFbeta pathway. The methods include administering one or more inhibitory nucleic acids targeting Trem2 and/or ApoE), sense nucleic acids encoding Egr1 and/or Mertk, and/or antibodies that bind to and inhibit Trem2 and/or ApoE.

### CLAIM OF PRIORITY

This application claims the benefit of U.S. Provisional Application Ser. No. 62/065,876, filed on Oct. 20, 2014, and 62/080,628 filed on Nov. 17, 2014. The entire contents of the foregoing are incorporated herein by reference.

### FEDERALLY SPONSORED RESEARCH OR DEVELOPMENT

This invention was made with Government support under Grant No R01NS088137 awarded by the National Institutes of Health. The Government has certain rights in the invention.

### TECHNICAL FIELD

Described herein are methods for treating neurologic diseases, e.g., amyotrophic lateral sclerosis (ALS) and multiple sclerosis, by modulating the APOE-TGFbeta pathway. The methods include administering one or more inhibitory nucleic acids targeting Trem2 and/or ApoE), sense nucleic acids encoding Egr1 and/or Mertk, and/or antibodies that bind to and inhibit Trem2 and/or ApoE.

### BACKGROUND

Inflammation has been implicated in a number of neurodegenerative disorders (e.g., amyotrophic lateral sclerosis (ALS) and multiple sclerosis). For example, increased inflammatory responses have been observed in both human ALS patients and animal models of ALS (McGreer et al., Muscle Nerve 26:459-470, 2002; Beers et al., Proc. Natl. Acad. Sci. U.S.A. 105:15558-15563, 2008; Banerjee et al., PLoS ONE 3:e2740, 2008; Chiu et al., Proc. Natl. Acad. Sci. U.S.A. 105:17913-17918, 2008; Chiu et al., Proc. Natl. Acad. Sci. U.S.A. 106:20960-20965, 2009; Beers et al., Proc. Natl. Acad. Sci. U.S.A. 103:16021-16026, 2006; Henkel et al., Ann. Neurol. 55:221-235, 2004; Meissner et al., Proc. Natl. Acad. Sci. U.S.A. 107:13046-13050, 2010). It has been reported that both microglia and astrocytes are activated in the central nervous system in a mouse model of familial ALS (Alexianu et al., Neurology 57:1282-1289, 2001; Hall et al., Glia 23:249-256, 1998), and that natural killer cells and peripheral T-cells infiltrate the spinal cord during neurodegenerative disease progression in a mouse model of ALS (Chiu et al., Proc. Natl. Acad. Sci. U.S.A. 105:17913-17918, 2008).

Microglia not only phagocytose cellular debris and apoptotic neurons, but, once activated, they might also engulf stressed but living neurons (Brown and Neher, Nat Rev Neurosci. 2014 April; 15(4):209-16). Evidence for this subtype of conventional phagocytosis that has been termed “phagoptosis” is cumulating from in vitro studies (Neniskyte et al., Journal of Biological Chemistry 286:39904-39913 (2011). Epub 2011 Sep. 8) and different mouse models including stroke (Neher et al., Proc Natl Acad Sci USA. 2013 October 22; 110(43): E4098-E4107. Epub 2013 Oct. 7), retinal degeneration (Zhao et al., EMBO Molecular Medicine 7(9):1179-1197, September 2015. Epub ahead of print), and LPS induced neuroinflammation (Fricker et al., J Neurosci. 2012 Feb. 22; 32(8):2657-66).

### SUMMARY

Described herein is a new pathway related to neurodegeneration in ALS, which provides a new avenue to specifically immune modulate microglia in ALS. Trem2 was identified as the executing microglial receptor in the upregulation of APOE upon phagocytosis of apoptotic neurons. Without wishing to be bound by theory, targeting APOE restored TGFbeta-dependent homeostatic signatures and its downstream targets including Mertk and Egr1, which were identified as negative-feedback regulators of APOE pathway. Thus, described herein are methods of treating neurodegenerative disease, e.g., ALS, by inhibiting ApoE and/or Trem2, and/or by increasing the activity and/or expression of Mertk2 and/or Egr1.

Thus provided herein are inhibitors of Trem2 or Apoe, e.g., inhibitory nucleic acids comprising a sequence that is complementary to a contiguous sequence present in Trem2 and/or ApoE, e.g., a contiguous sequence of at least 10 nucleotides, and/or small molecules or antibodies that bind to and inhibits Trem2 or ApoE, for use in treating amyotrophic lateral sclerosis (ALS) in a subject.

Also provided herein are methods for treating amyotrophic lateral sclerosis (ALS) in a subject that include administering to a subject having ALS a therapeutically effective amount of at least one inhibitor of Trem2 or Apoe, e.g., inhibitory nucleic acids targeting Trem2 and/or ApoE, and/or at least one antibody that binds to and inhibit Trem2 or ApoE.

In some embodiments, the at least one inhibitory nucleic acid is an antisense oligonucleotide or small interfering RNA.

Also provided herein are sense nucleic acid encoding Egr1 and/or Mertk for use in treating amyotrophic lateral sclerosis (ALS) in a subject.

Further, provided herein are methods for treating amyotrophic lateral sclerosis (ALS) in a subject that include administering to a subject having ALS a therapeutically effective amount of at least one sense nucleic acid encoding Egr1 and/or Mertk, e.g., in a viral vector such as an AAV.

In some embodiments, the inhibitory or sense nucleic acid or antibody is injected into the cerebrospinal fluid of a subject.

In some embodiments, the nucleic acid or antibody is administered by intracranial injection or intrathecal injection.

In some embodiments, the at least one inhibitory nucleic acid is complexed with one or more cationic polymers and/or cationic lipids.

As used herein, “RNA” refers to a molecule comprising at least one or more ribonucleotide residues. A “ribonucleotide” is a nucleotide with a hydroxyl group at the 2′ position of a beta-D-ribofuranose moiety. The term RNA, as used herein, includes double-stranded RNA, single-stranded RNA, isolated RNA, such as partially purified RNA, essentially pure RNA, synthetic RNA, recombinantly-produced RNA, as well as altered RNA that differs from naturally-occurring RNA by the addition, deletion, substitution and/or alteration of one or more nucleotides. Nucleotides of the RNA molecules can also comprise non-standard nucleotides, such as non-naturally occurring nucleotides or chemically synthesized nucleotides or deoxynucleotides.

By the term “increase” is meant an observable, detectable, or significant increase in a level as compared to a reference level or a level measured at an earlier or later time point in the same subject.

By the term “decrease” is meant an observable, detectable, or significant decrease in a level as compared to a reference level or a level measured at an earlier or later time point in the same subject.

By the term “neurodegenerative disorder” is meant a neurological disorder characterized by a progressive loss of neuronal function and structure, and neuron death. Non-limiting examples of neurodegenerative disorders include Parkinson's disease (PD), Alzheimer's disease (AD), Huntington's disease (HD), brain stroke, brain tumors, cardiac ischemia, age-related macular degeneration (AMD), retinitis pigmentosa (RP), amyotrophic lateral sclerosis (ALS, e.g., familial ALS and sporadic ALS), and multiple sclerosis (MS). Methods for diagnosing a neurodegenerative disorder are described herein. Additional methods for diagnosing a neurodegenerative disorder are known in the art. In some embodiments, the present methods exclude AD.

By the term “inhibitory RNA” is meant a nucleic acid molecule that contains a sequence that is complementary to a target nucleic acid (e.g., TREM2 or APOE) that mediates a decrease in the level or activity of the target nucleic acid (e.g., activity in CD14⁺CD16⁻ or CD14⁺CD16⁺ monocyte). Non-limiting examples of inhibitory RNAs include interfering RNA, shRNA, siRNA, ribozymes, antagomirs, and antisense oligonucleotides. Methods of making inhibitory RNAs are described herein. Additional methods of making inhibitory RNAs are known in the art.

As used herein, “an interfering RNA” refers to any double stranded or single stranded RNA sequence, capable—either directly or indirectly (i.e., upon conversion)—of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

As used herein “an shRNA” (small hairpin RNA) refers to an RNA molecule comprising an antisense region, a loop portion and a sense region, wherein the sense region has complementary nucleotides that base pair with the antisense region to form a duplex stem. Following post-transcriptional processing, the small hairpin RNA is converted into a small interfering RNA by a cleavage event mediated by the enzyme Dicer, which is a member of the RNase III family.

A “small interfering RNA” or “siRNA” as used herein refers to any small RNA molecule capable of inhibiting or down regulating gene expression by mediating RNA interference in a sequence specific manner. The small RNA can be, for example, about 18 to 21 nucleotides long.

As used herein, the phrase “post-transcriptional processing” refers to mRNA processing that occurs after transcription and is mediated, for example, by the enzymes Dicer and/or Drosha.

By the phrase “risk of developing disease” is meant the relative probability that a subject will develop a neurodegenerative disorder in the future as compared to a control subject or population (e.g., a healthy subject or population). Provided herein are methods for reducing a subject's risk of developing a neurodegenerative disease in the future.

By the phrase “rate of disease progression” is meant one or more of the rate of onset of symptoms of a neurodegenerative disorder in a subject, the rate of the increasing intensity (worsening) of symptoms of a neurodegenerative disorder in a subject, the frequency of one or more symptoms of a neurodegenerative disorder in a subject, the duration of one or more symptoms of a neurodegenerative disorder in a subject, or the longevity of subject. For example, an increased rate of disease progression can include one or more of: an increased rate of onset of symptoms of a neurodegenerative disorder in a subject, an increased frequency of one or more symptoms of a neurodegenerative disorder in a subject, an increase in the duration of one or more symptoms of a neurodegenerative disorder in a subject, or a decrease in the longevity of a subject. Methods of predicting the rate of disease progression in a subject having a neurodegenerative disorder are described herein.

By the term “purifying” is meant a partial isolation of a substance from its natural environment (e.g., partial removal of contaminating biomolecules or cells). For example, a monocyte (e.g., a CD14⁺CD16⁻ or CD14⁺CD16⁺ monocyte) can be purified from other cell types present in a sample of peripheral blood (e.g., using fluorescence-assisted cell sorting).

The term “treating” includes reducing the number of symptoms or reducing the severity, duration, or frequency of one or more symptoms of disease (e.g., a neurodegenerative disease) in a subject. The term treating can also delaying the onset or progression of symptoms, or progression of severity of symptoms, of a neurodegenerative disorder in a subject, or increasing the longevity of a subject having a neurodegenerative disorder.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-E. Upregulation of APOE and downregulation of TGFbeta1 signaling is a common pathway in disease-associated microglia. Extensive profiling of gene expression of selected microglia specific and proinflammatory genes was performed by MG400 and mouse inflammation Nanostring chip from disease-associated microglia of brains or spinal cords from mouse models of AD (APP/PS1), MS (EAE) and ALS (SOD1) (N=3). (a) Venn diagram summarizes Nanostring chip analysis results displaying common and unique microglial genes dys-regulated in the three diseases. (b) Common 50 genes that are significantly dys-regulated in disease associated microglia. Apoe is the top upregulated gene in all three investigated diseases. Bars show expression fold change compared to naïve microglia of age matched controls (N=3/investigated disease) p<0.05. (c) and (d) In EAE, microglia homeostatic signature is severely suppressed in diseases stages in brain and spinal. Of note, changes were most significant at acute and chronic disease. In contrast, microglia inflammatory signature is drastically upregulated including Apoe. Bars show absolute mRNA counts per 100 ng total RNA compared to naïve microglia (N=3) p<0.05, Student t test, 2-tailed. (e) Common transcription regulation in microglia in disease: Ingenuity pathway analysis shows common nodes significantly affected in microglia in all three investigated diseases. TGFb and APOE are central in the disease associated signaling axis. For each molecule in the data set, the expression fold change compared to normal, healthy microglia is presented. The legend shows prediction state and relationships.

FIGS. 2A-G. Phagocytosis of apoptotic neurons leads to disease-associated phenotype in microglia. Representative fluorescent cell sorting (FACS) analysis of microglia stained with FCRLS (resident microglia) and CD11b (microglia/monocytes). Phagocytic microglia is further purified from this cell population by the abundance of the apoptotic neuron labeling fluorophore Alexa488. In contrast, non-phagocytic microglia from the same brain does not contain Alexa488 labeling. (b) Immunohistochemical staining of the brain injection site for apoptotic neurons versus PBS only injection. Representative images stained with the microglia/monocyte marker Iba1 and the microglia specific P2ry12 are shown (N=6). Apoptotic neurons attract microglia to the site of injection in contrast to PBS (c) Confocal microscopy using brain resident microglia specific antibody P2ry12 confirmed attraction of microglia to the site of apoptotic neuron injection. Orthogonal projections of confocal z-stacks show intracellular phagocytosed dead neurons and neuronal debris within microglia (N=3) (scale bar: 5 μm). (d) Extensive profiling of gene expression of selected microglia specific molecules and proinflammatory genes was performed by MG468 Nanostring chip (N=3). Heat map of significantly affected genes in apoptotic neuron-phagocytic versus non-phagocytic microglia showed widespread changes in microglia signature after phagocytosis with upregulation of Apoe and proinflammatory molecules (Ccl2, IL1b, Nos2 and others in red) and loss of microglia homeostatic signature (Tmem119, Trem2, P2ry12 and others in blue); p<0.05, Student t test, 2-tailed. (e) TOP-40 upregulated as well as downregulated genes in apoptotic neuron-phagocytic microglia determined by MG468 chip analysis are shown in comparison with non-phagocytic microglia from the same brain. Of note, Apoe is one of the most upregulated genes in phagocytic microglia. Bars show absolute mRNA counts (N=3). (f) qPCR validation of selected target genes confirmed MG468 Nanostring chip analysis and showed substantial upregulation of Apoe and other proinflammatory genes including miR155 and widespread downregulation of microglia homeostasis genes like P2ry12 and TGFbr1 in apoptotic neuron phagocytic microglia. Of note, non-phagocytic microglia from the same brain and microglia sorted form PBS-injected brains did not upregulate inflammatory signaling genes and only marginally downregulated microglia homeostasis genes; qPCRs were run in duplicates; bars show relative expression. The experiment was conducted independently 7 times with identical results. Data represent 5 mice per experimental groups (mean±standard error of the mean; 1-way analysis of variance; Kruskal-Wallis multiple comparisons test) p<0.05. (g) Ingenuity pathway analysis of apoptotic neuron-phagocytic microglia shows affected genes and their potential connections in the APOE-TGFb-signaling axis. For each molecule in the data set, the expression fold changes as compared to non-phagocytic microglia from the same brains are shown.

FIGS. 3A-H. Suppression of the homeostatic molecular signature in phagocytic microglia is regulated by APOE pathway but independent from miR155 pathway. Expression profiling of phagocytic microglia from WT versus Apoe-KO or miR155-KO mice by MG468 Nanostring chip revealed significant differences in gene activation pointing to different functions of Apoe and miR155 pathway. (a)+(b) Top-up- and -down regulated genes in phagocytic microglia from Apoe-KO mice as detected by nCounter profile. Of note, microglia homeostasis genes are much less suppressed in phagocytic microglia from Apoe-KO mice compared to WT (N=3). The data are shown as mRNA count per 100 ng of total RNA. (c) qPCR validation of selected target genes confirmed MG468 Nanostring chip analysis and showed that microglia homeostasis genes are less down regulated in phagocytic Apoe-KO microglia compared to WT. qPCRs were run in duplicates; bars show relative expression (N=4 per group). (d) miR155 expression is significantly suppressed in Apoe-KO microglia after phagocytosis of apoptotic neurons. In contrast, upregulation of Apoe expression upon phagocytosis is unchanged in miR155-KO microglia. qPCRs were run in duplicates; bars show relative expression (N=3 per group). (e) VENN diagram show non-overlapping pathways of microglia activation by phagocytosis in Apoe-KO and miR155-KO mice. (f) Top-10 affected genes highlight different functional pathways of Apoe and miR155 signaling. (g) Whereas Apoe is associated with microglia homeostasis, miR155 is clearly linked to inflammatory signaling. (h) Top regulator is TGFb1 in the Apoe-pathway in contrast to 116 in miR155 as determined by Ingenuity pathway analysis.

FIGS. 4A-I. Mertk (via Egr1) suppresses APOE pathway in homeostatic microglia. (a) Apoe, Mertk and Egr1 are tightly and reciprocally regulated during development (data taken from NN) (b) Vulcano plot of Nanosting MG550 analysis of naïve brain derived adult Egr1−/− microglia versus WT showed downregulation of homeostasis genes including Mertk and upregulation of Apoe expression (N=3) (c) qPCR analysis confirmed significant upregulation of Apoe in naïve Egr1−/− microglia. (N=3 per group) (d) Adult microglia cells sorted from WT or Mertk−/− brains were cultured in vitro and treated with LPS. qPCR was run to determine Apoe expression level (N=3). (e) The expression of microglia key genes in Mertk−/− and Axl−/− microglia was intensively profiled in comparison to WT microglia using MG468 chip. Heatmap of naïve microglia from WT, Axl−/− and Mertk−/− mice showed downregulation of homeostatic signature in Mertk−/−. In contrast, this signature is unchanged by Axl (N=4/group) (f) Correspondence analysis of samples (large spheres) and genes (small spheres) (g)+(h) Volcano plots based on NanoString gene expression data comparing microglia transcripts from Mertk−/− or Axl−/− versus WT, respectively. Red dots show significantly up-, whereas blue dots significantly down regulated genes in Mertk−/− and Axl−/− versus WT microglia (p<0.05 by Student t test, 2-tailed) (i) qPCR validation of selected target genes confirmed MG468 Nanostring chip analysis and showed substantial upregulation of Apoe in naïve Mertk−/− microglia, that was not changed upon phagocytosis (N=4/group) (p<0.05 by Student t test, 2-tailed).

FIGS. 5A-F. Genetic ablation of TREM2 significantly suppresses APOE pathway and restores the homeostatic genes in both WT and APP/PS1 mice. The regulation of microglia homeostatic genes were intensively profiled in Trem2-KO versus WT mice in naïve and disease conditions. (a) Heat map of naïve microglia from WT and Trem2-KO mice showed widespread changes in expression level of key genes in Trem2-KO microglia. Each lane represents one sample (N>3 per group). (b) Volcano plot based on Nanostring gene expression analysis highlight significant changes in Trem2-KO (p<0.05 by Student t test, 2-tailed). (c) Top-up and -down regulated genes in Trem2-KO microglia demonstrate widespread enhancement of the microglia homeostatic signature (p<0.05 by Student t test, 2-tailed). (d) Comparison of expression signature in disease (APP-PS1) by heat map analysis showed intensive influence of Trem2-KO on microglia homeostasis. (e) Volcano plot based on Nanostring gene expression analysis summarizes significant changes in Trem2-KO microglia in disease compared to WT. (f) Top-up and -down regulated genes in Trem2-KO microglia in disease demonstrate widespread and significant reset of the microglia homeostatic signature compared to loss of homeostatic signature in WT in APP/PS1-mice. Of note, genes significantly upregulated in disease associated WT microglia including Apoe, Axl and Csf1 are unaffected in Trem2-KO microglia (p<0.05 by Student t test, 2-tailed).

FIGS. 6A-D. Microglia signature changes during EAE disease course. (a) Spinal cords were collected at 3 different disease stages during EAE. (b) CD11b+/FCRLS+/Ly6C−-Microglia was sorted from spinal cords and (c) intensively profiled using MG400 and mouse Inflammation Nanostring expression chip. In comparison to naïve microglia, homeostatic signature is severely downregulated in microglia at acute stages and highly compromised in recovery and chronic disease. Inflammatory molecules are upregulated in all EAE stages. Misregulated key molecules are highlighted in blue (homeostasis genes) and red (inflammatory genes). (d) Significantly affected microglial genes in different EAE stages grouped according to cell localization or function. Bars show expression fold changes compared to naïve microglia (N=3).

FIGS. 7A-C. AD-associated microglia signature. CD11b+/FCRLS+/Ly6C−-microglia were sorted from brain and intensively profiled using MG400 and mouse Inflammation Nanostring expression chip. (a) Significantly affected microglial genes in 1 year old APP/PS1-mice grouped according to cell localization or function. Bars show expression fold changes compared to naïve microglia from age matched WT mice (N=3). (b) Top-10 downregulated and (c) Top-10 upregulated genes in APP/PS1-mice compared to age matched WT-mice as detected by nCounter profile. Bars in b+c shown mRNA count per 100 ng of total RNA.

FIG. 8. SOD1-associated microglia signature. CD11b+/FCRLS+/Ly6C−-microglia were sorted from brain of clinical mice and intensively profiled using MG400 and mouse Inflammation Nanostring expression chip. Graphs were generated from published datasets (2015 AoN). Significantly affected microglial genes grouped according to cell localization or function. Of note, global metabolism of microglia is suppressed in SOD1-mice with widespread downregulation of most microglial genes. Bars show expression fold changes compared to naïve microglia from age matched WT mice (N=3).

FIG. 9. Unique affected microglial genes in mouse models of neurodegenerative and neuroinflammatory diseases. Extensive expression profiling of unique and enriched microglia specific genes was performed by MG400 Nanostring chip. Genes are displayed that are misregulated in the individual diseases only. Bars show absolute mRNA counts (N=3/investigated disease).

FIG. 10. Apoe expression is highly upregulated in microglia in acute and chronic stages in different EAE models. Severe upregulation of Apoe expression in different disease stages in two alternative EAE models underline the universal role of Apoe in microglia in disease. qPCRs were run in duplicates; bars show relative expression of representative genes compared to naïve microglia (N=3/disease stage) p<0.05, Student t test, 2-tailed.

FIGS. 11A-B. M1 polarization of microglia in vitro and in vivo does not lead to increase of Apoe expression. (a) Polarization of adult mouse microglia in vitro to classical M1 (+LPS, IFNg) or M2 (+IL4) does not lead to an increased Apoe expression. In contrast, M1 lead to severe and significant down regulation of Apoe expression. Bars show relative expression determined by qPCR (N=4) (b) Stereotactic injection of LPS to brains of adult mice stimulated expression of proinflammatory cytokines like Il1b and TNFalpha by microglia, but again failed to induce expression of Apoe. Bars show absolute mRNA counts per 100 ng total RNA compared to microglia from PBS injected brains (N=3) p<0.05, Student t test, 2-tailed.

FIGS. 12A-E. Microglia efficiently phagocytose apoptotic neurons but not live or necrotic neurons. Apoptotic neurons are efficiently phagocytosed by microglia in contrast to live or necrotic neurons. (a) After injection of apoptotic neurons to the brain, these are efficiently phagocytosed by microglia within 16 h. Microglia cells were first FACS sorted with FCRLS (resident microglia) and CD11b (microglia/monocytes). The phagocytic population was separated from non-phagocytic microglia via abundance of Alexa488, the fluorophore used to label dead neurons. In contrast, after injection of live neurons, only a very small subset of the microglia population exhibits phagocytosis. (b) Quantification of the phagocytosis efficiency revealed that live neurons are no target for phagocytosis by microglia; in contrast, apoptotic neurons are efficiently phagocytosed; p<0.001, Student t test, 2-tailed (N=5). (c) Representative FACS sorting data demonstrated that the efficiency of phagocytosis of necrotic neurons is likewise reduced compared to apoptotic neurons. (d) Quantification of the phagocytosis efficiency showed significantly less phagocytosis of necrotic in contrast to apoptotic neurons by microglia; p<0.05, Student t test, 2-tailed (N=5). (e) Significant upregulation of Apoe was seen in microglia phagocytosing apoptotic monocytes.

FIG. 13. Immunohistochemical analysis show widespread microglia activation and neuronal loss after injection of apoptotic neurons. 16 h post injection of apoptotic neurons or PBS as control, brains of mice were subjected to histological or immunohistochemical staining and representative pictures are shown here for either injection site. Only after injection of neurons, widespread microglia recruitment could be detected (see Iba1 for microglia/monocytes or 4D4 and P2ry12 for brain resident microglia). Moreover, this seem to be accompanied with considerable neuronal loss (NeuN staining). Activation of caspase 3 could be likewise only detected in the dead neuron injected samples (Act. Caspase). In contrast, staining for oligodendrocytes (CNPase) or oligodendrocyte precursor cells (NG2) did not reveal differences. Discuss GFAP and APOE?? (N=6/group).

FIGS. 14A-B. Phagocytosis of apoptotic neurons trigger increase of Apoe expression in microglia within 16 hours. Apoptotic neurons were injected to the brain and microglia were FACS sorted 3, 8 or 16 h later. PBS injection served as control. Microglia populations were further sorted for phagocytosis of labeled neurons and analyzed by qPCR for expression of (a) miR155 and (b) Apoe. miR155 and Apoe are both significantly upregulated 16 h post injection in phagocytic microglia. Non-phagocytic microglia and PBS-control-microglia did not significantly upregulate either; (N=3/group) p<0.05, Student t test, 2-tailed.

FIGS. 15A-B. Upregulation of Apoe expression in microglia is specific for phagocytosis of apoptotic neurons. To address the question whether upregulation of Apoe together with the loss of homeostatic signature in microglia is specific for the phagocytosis of apoptotic neurons, we stereotactically injected labeled E coli, Zymosan or apoptotic neurons into the brain of WT mice and FACS sorted microglia 16 h later. All microglia populations were further sorted for uptake of labeled material and analyzed by qPCR for (a) Apoe or (b) miR155. Importantly, only the phagocytosis of apoptotic neurons led to an increase in Apoe expression, whereas all three materials led to significant upregulation of miR155; (N=3/group) p<0.05, Student t test, 2-tailed.

FIGS. 16A-B. Neurons do not contribute to Apoe expression of phagocytic microglia. (a) qPCR analysis confirmed that key genes which are upregulated in phagocytic microglia (MG-Φ) are not at all or only mildly expressed in primary neurons (Live neurons). After induction of apoptosis in neurons (Apoptotic neurons 2 h), expression levels of those genes in neurons even decrease and are completely undetectable after additional 16 h incubation (Apoptotic neurons 18 h). Bars show relative expression (N=3/group), p<0.0001, Student t test, 2-tailed. (b) Phagocytosis of apoptotic neurons derived from Apoe-KO-mice by microglia significantly upregulated Apoe-expression in phagocytic microglia and demonstrated that this increase of Apoe expression is derived from phagocytic microglia only while neuronal RNAs do not contribute to the detected changes. Bars show relative expression (N=3/group), Student t test, 2-tailed.

FIGS. 17A-B. AnnexinV blocks phagocytosis of apoptotic neurons by microglia. To identify the ligand for phagocytosis by microglia on apoptotic neurons, we pretreated apoptotic neurons with AnnexinV, an established blocker of phosphatidylserine. The latter is exposed on the outer leaflet of the cellular membrane upon induction of apoptosis. (a) Representative FACS sorting plot showed severely reduced numbers of phagocytic microglia after pretreatment of neurons with AnnexinV. (b) Quantification of phagocytosis efficiency revealed that pretreatment of apoptotic neurons with AnnexinV almost completely blocked uptake by microglia (N=4), p<0.001, Student t test, 2-tailed.

FIGS. 18A-C. (a) Venn diagram showing common and unique identified genes upregulated or downregulated in 1- and MGnd-induced adult mouse brain microglia. (b) Unique molecular signature of adult mouse brain M1 microglia. (c) Unique molecular signature of adult mouse brain MGnd microglia.

### DETAILED DESCRIPTION

Microglia are the resident immune phagocytes of CNS (1). They migrate into the developing CNS during early embryogenesis and then proliferate as a CNS endogenous cell population distinct from all other tissue macrophages and circulating monocytes (Ginhoux et al., Science. 2010 Nov. 5; 330(6005):841-5. Epub 2010 Oct. 21; Kierdorf et al., Nat Neurosci. 2013 March; 16(3):273-80. Epub 2013 Jan. 20; Schulz et al., Science. 2012 Apr. 6; 336(6077):86-90. Epub 2012 Mar. 22). Indeed, adult microglia have been recently identified in the healthy brain as presenting a unique molecular signature characterized by the expression of key proteins e.g. TGFbeta1 P2ry12 Hexb Tam Receptors system etc (Butovsky et al., Nat Neurosci. 2014 January; 17(1):131-43. Epub 2013 Dec. 8). Microglia main role is to constantly survey their environment (Nimmerjahn et al., Science. 2005 May 27; 308(5726):1314-8). They are believed to act as sensors during brain development to shape neuronal connectivity (2) and also to react to invading pathogens (3) and cellular debris including protein aggregates or dying cells by setting up the so-called inflammatory reaction. They start secreting effectors molecules from cytokines to chemokines and end the reaction by phagocyting or endocyting homeostasis-perturbating elements in order to clean up and maintain brain physiology (4).

Phagocytosis of apoptotic neurons by microglia is thought to be initiated by the exposure of so called “eat-me” signals on the neuronal membrane (Ravichandran, Immunity 2011 Oct. 28; 35(4):445-55) such as phosphatidylserine, calreticulin or complement factors. Microglia express a couple of different receptors to interact with these signaling cues. These include proteins of the TAM-family of receptor tyrosine kinases (MERTK and AXL) in concert with the adaptor proteins Gas6 or Pros 1 (Scott et al., Nature. 2001 May 10; 411(6834):207-11), Lipoprotein Receptor-related Protein 1 (LRP1) and others (Brown and Neher, Trends Biochem Sci. 2012 August; 37(8):325-32). In addition, Triggering Receptor Expressed on Myeloid Cells 2 (Trem2) has been implicated in the removal of cellular and myelin debris in the brain (Kleinberger et al., Sci Transl Med. 2014 Jul. 2; 6(243):243ra86; Wang et al., Cell. 2015 Mar. 12; 160(6):1061-71. Epub 2015 Feb. 26). Trem2 is preferentially expressed on microglia cells and missense mutations in Trem2 have been identified as a risk factor for a couple of neurodegenerative diseases including AD, ALS, Parkinson's disease and frontotemporal dementia (Kleinberger et al., Sci Transl Med. 2014 Jul. 2; 6(243):243ra86), leading others to suggest the use of Trem2 activators for AD and other diseases (Wang et al., Cell. 2015 Mar. 12; 160(6):1061-71. Epub 2015 Feb. 26).

When microglia are chronically activated in the course of neuroinflammatory and/or neurodegenerative diseases, they lose their beneficial abilities to restore homeostasis and as a consequence acquire a detrimental molecular and functional phenotype and may also contribute to further neuronal death (Zhang et al., Cell. 2013 April 25; 153(3):707-20). However, until now, how this switch is executed on the molecular level has been poorly understood. As shown herein, a common pattern of microglia dysfunction was identified that was associated with diverse CNS disease mouse models including Multiple Sclerosis (MS), Amyothrophic lateral sclerosis (ALS) and Alzheimer's Disease (AD). This disease associated expression pattern was characterized by loss of a microglia homeostatic signature with downregulation of key molecules like P2ry12, Csf1r, Mertk and Tgfb1. Surprisingly, the most upregulated gene in disease-associated microglia was Apolipoprotein E (APOE). MERTK, AXL and Trem2 knock-out and microglial LRP1 conditional knockout mice were used to elucidate the impact of these receptors on the APOE-TGFb signaling axis upon phagocytosis of apoptotic neurons in vivo. The MERTK-KO microglia homeostatic signature was already downregulated in resting microglia, suggesting a main function of MERTK to sustain microglia homeostasis in the healthy brain. Interestingly, knockout of AXL, LRP1 and MERTK had no impact on the phagocytosis efficiency of apoptotic neurons and lead to disease-associated microglia phenotype comparable to WT. Of note, APOE pathway is not upregulated in microglia of Trem2-Ko mice upon phagocytosis of apoptotic neurons. Thus, it was hypothesized that apoptotic neurons engage TREM2 via exposed lipids and activate the downstream APOE pathway.

The role of Trem2 in microglia has been controversial. Although the rare TREM2 R47H mutation is known to confer high risk for AD (Kleinberger, G., et al. Sci. Transl. Med. 6, 243ra86 (2014))(5, 6), TREM2's exact role in the disease is still unclear. Several murine studies suggest a beneficial role for TREM2 in reactive microgliosis (Wang et al., Cell. 2015 Mar. 12; 160(6):1061-71), suppressing inflammation (Wang et al., Cell. 2015 Mar. 12; 160(6):1061-71; Jiang, T., et al. Neuropsychopharmacology 39, 2949-2962 (2014)), and promoting phagocytosis of amyloid beta and apoptotic neurons (Kleinberger, G., et al. Sci. Transl. Med. 6, 243ra86 (2014); Jiang, T., et al. Neuropsychopharmacology 39, 2949-2962 (2014)). In contrast, the present results support a pathogenic role for increased TREM2 expression by peripherally-derived myeloid cells in AD susceptibility.

In summary, we have identified the APOE-TGFB axis as a critical common regulatory pathway in microglia. This pathway is dysregulated in both inflammatory and degenerative diseases of the CNS, initiated by the recognition and phagocytosis of apoptotic neurons via membrane-exposed phosphatidylserine and probably executed by Trem2. Thus, described herein are methods of treating a neurodegenerative disorder (e.g., ALS or MS) that include administering to a subject at least one agent that decreases the level or activity of one or more of ApoE and/or TREM2, and/or at least one agent that increases the level or activity of one or more of Mertk and/or Egr1.

### Neurodegenerative Disorders

Neurodegenerative disorders are a class of neurological diseases that are characterized by the progressive loss of the structure and function of neurons and neuronal cell death. Inflammation has been implicated for a role in several neurodegenerative disorders. Progressive loss of motor and sensory neurons and the ability of the mind to refer sensory information to an external object is affected in different kinds of neurodegenerative disorders. Non-limiting examples of neurodegenerative disorders include Parkinson's disease, Alzheimer's disease, Huntington's disease, amyotrophic lateral sclerosis (ALS, e.g., familial ALS and sporadic ALS), and multiple sclerosis (MS). In some embodiments, the neurodegenerative disorder is not Alzheimer's disease.

A health care professional may diagnose a subject as having a neurodegenerative disorder by the assessment of one or more symptoms of a neurodegenerative disorder in the subject. Non-limiting symptoms of a neurodegenerative disorder in a subject include difficulty lifting the front part of the foot and toes; weakness in arms, legs, feet, or ankles; hand weakness or clumsiness; slurring of speech; difficulty swallowing; muscle cramps; twitching in arms, shoulders, and tongue; difficulty chewing; difficulty breathing; muscle paralysis; partial or complete loss of vision; double vision; tingling or pain in parts of body; electric shock sensations that occur with head movements; tremor; unsteady gait; fatigue; dizziness; loss of memory; disorientation; misinterpretation of spatial relationships; difficulty reading or writing; difficulty concentrating and thinking; difficulty making judgments and decisions; difficulty planning and performing familiar tasks; depression; anxiety; social withdrawal; mood swings; irritability; aggressiveness; changes in sleeping habits; wandering; dementia; loss of automatic movements; impaired posture and balance; rigid muscles; bradykinesia; slow or abnormal eye movements; involuntary jerking or writhing movements (chorea); involuntary, sustained contracture of muscles (dystonia); lack of flexibility; lack of impulse control; and changes in appetite. A health care professional may also base a diagnosis, in part, on the subject's family history of a neurodegenerative disorder. A health care professional may diagnose a subject as having a neurodegenerative disorder upon presentation of a subject to a health care facility (e.g., a clinic or a hospital). In some instances, a health care professional may diagnose a subject as having a neurodegenerative disorder while the subject is admitted in an assisted care facility. Typically, a physician diagnoses a neurodegenerative disorder in a subject after the presentation of one or more symptoms.

Provided herein are additional methods for diagnosing a neurodegenerative disorder in a subject (e.g., a subject presenting with one or more symptoms of a neurodegenerative disorder or a subject not presenting a symptom of a neurodegenerative disorder (e.g., an undiagnosed and/or asymptomatic subject). Also provided herein are prognostic methods and methods of treating a neurodegenerative disorder in a subject (e.g., methods of decreasing the rate of onset or the progression of symptoms (e.g., ataxia) of a neurodegenerative disorder in a subject).

### Methods of Treatment

Also provided are methods of treating a neurodegenerative disorder (e.g., ALS or MS) that include administering to a subject at least one agent that decreases the level or activity of one or more of ApoE and/or TREM2, and/or increases the level or activity of one or more of Mertk and/or Egr1. In some embodiments, the subject is first identified or selected for treatment using any diagnostic methods known in the art.

Useful sequences for these genes and proteins are known in the art. Exemplary human sequences are provided in Table A:

TABLE A
EXEMPLARY HUMAN mRNA AND PROTEIN SEQUENCES

| GenBank | GenBank | |
|---|---|---|
| Acc. No.-mRNA | Acc. No.-protein | Gene name |
| NM_006343.2 | NP_006334.2 | tyrosine-protein kinase Mer |
| | | precursor (Mertk) |
| NM_001964.2 | NP_001955.1 | early growth response |
| | | protein 1 (Egr1) |
| Variant 1 | Variant 1 | Triggering receptor |
| NM_018965.3 | NP_061838.1 | expressed on myeloid cells |
| Variant 2 | Variant 2 | 2 (TREM2) |
| NM_001271821.1 | NP_001258750.1 | |
| NM_000041.3 | NP_000032.1 | apolipoprotein E isoform b |
| | | precursor |
| NM_001302688.1 | NP_001289617.1 | apolipoprotein E isoform a |
| | | precursor |
| NM_001302689.1 | NP_001289618.1 | apolipoprotein E isoform b |
| | | precursor |
| NM_001302690.1 | NP_001289619.1 | apolipoprotein E isoform b |
| | | precursor |
| NM_001302691.1 | NP_001289620.1 | apolipoprotein E isoform b |
| | | precursor |

In some embodiments, the agent that decreases the level or activity of one or more of ApoE and/or TREM2 is an inhibitory nucleic acid; for example, the subject can be administered at least one inhibitory nucleic acid comprising a sequence that is complementary to a contiguous sequence present in ApoE and/or at least one inhibitory nucleic acid comprising a sequence that is complementary to a contiguous sequence present in TREM2. In non-limiting embodiments, the inhibitory nucleic acid can be an antisense oligonucleotide, a ribozyme, or an siRNA. In some embodiments, the at least one inhibitory nucleic acid is injected into the cerebrospinal fluid of a subject. In some embodiments, the injection is intracranial injection or intrathecal injection. In some embodiments, the at least one inhibitory nucleic acid is complexed with one or more cationic polymers and/or cationic lipids (e.g., any of the cationic polymers described herein or known in the art). Inhibitory nucleic acids to decrease the expression and/or activity of a specific target mRNA (e.g., ApoE or TREM2) can be designed using methods known in the art (see, e.g., Krutzfeld et al., Nature 438:685-689, 2005). Additional exemplary methods for designing and making inhibitory nucleic acids are known in the art and described herein.

In some embodiments, the subject is administered at least one sense nucleic acid comprising a sequence that encodes Mertk or Egr1.

In some embodiments, the agent that decreases the level or activity of one or more of ApoE and/or TREM2 is an inhibitory antibody, or a small molecule inhibitor of ApoE or Trem2. In some embodiments, the APOE inhibitor is a soluble receptor for LDL (LDLR), such as the recombinant human LDL R 2148-LD/CF (R&D SYSTEMS). Methods for identifying additional inhibitory small molecules are known in the art; see, e.g., Wang et al., Cell. 2015 Mar. 12; 160(6):1061-71; WO2015110556; WO2000061069; WO 2013181618; and others.

A subject can be administered at least one (e.g., at least 2, 3, 4, or 5) dose of the agent (e.g., one or more inhibitory or sense nucleic acids, antibodies, peptides, or small molecules). The agent (e.g., one or more nucleic acids, antibodies, peptides, or small molecules) can be administered to the subject at least once a day (e.g., twice a day, three times a day, and four times a day), at least once a week (e.g., twice a week, three times a week, four times a week), and/or at least once a month. A subject can be treated (e.g., periodically administered the agent) for a prolonged period of time (e.g., at least one month, two months, six months, one year, two years, three years, four years, or five years). As described in detail herein, the dosage of the agent to be administered to the subject can be determined by a physician by consideration of a number of physiological factors including, but not limited to, the sex of the subject, the weight of the subject, the age of the subject, and the presence of other medical conditions. The agent can be administered to the subject orally, intravenously, intraarterially, subcutaneously, intramuscularly, intracranially, or via injection into the cerebrospinal fluid. Likewise, the agent may be formulated as a solid (e.g., for oral administration) or a physiologically acceptable liquid carrier (e.g., saline) (e.g., for intravenous, intraarterial, subcutaneous, intramuscular, cerebrospinal (intrathecal), or intracranial administration). In some embodiments, the agent (e.g., one or more inhibitory nucleic acids, antibodies, peptides, or small molecules) can be administered by injection or can be administered by infusion over a period of time.

The agents to be administered to a subject for treatment of a neurodegenerative disorder are described below, and can be used in any combination (e.g., at least one, two, three, four, or five of any combination of the agents or classes of agents described below).

### Inhibitory Nucleic Acids

Inhibitory agents useful in the methods of treatment described herein include inhibitory nucleic acid molecules that decrease the expression or activity of one or both of ApoE and/or TREM2.

Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds, such as siRNA compounds, modified bases/locked nucleic acids (LNAs), peptide nucleic acids (PNAs), and other oligomeric compounds, or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), mixmers, gapmers, or combinations thereof. See, e.g., WO 2010/040112.

In some embodiments, the inhibitory nucleic acids are 10 to 50, 13 to 50, or 13 to 30 nucleotides in length. One having ordinary skill in the art will appreciate that this embodies oligonucleotides having antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the oligonucleotides are 15 nucleotides in length. In some embodiments, the antisense or oligonucleotide compounds of the invention are 12 or 13 to 30 nucleotides in length. One having ordinary skill in the art will appreciate that this embodies inhibitory nucleic acids having antisense portions of 12, 13, 14, 15, 16, 17, 18, 19, 20, 21, 22, 23, 24, 25, 26, 27, 28, 29, or 30 nucleotides in length, or any range therewithin.

In some embodiments, the inhibitory nucleic acids are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides, and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5, 220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino, and 2′ O-methyl modifications on the ribose of pyrimidines, abasic residues, or an inverted base at the 3′ end of the RNA. Such modifications are routinely incorporated into oligonucleotides and these oligonucleotides have been shown to have a higher Tm (i.e., higher target binding affinity) than 2′-deoxyoligonucleotides against a given target.

A number of nucleotide and nucleoside modifications have been shown to make the oligonucleotide into which they are incorporated more resistant to nuclease digestion than the native oligodeoxynucleotide—the modified oligos survive intact for a longer time than unmodified oligonucleotides. Specific examples of modified oligonucleotides include those comprising modified backbones, for example, phosphorothioates, phosphotriesters, methyl phosphonates, short-chain alkyl or cycloalkyl intersugar linkages, or short-chain heteroatomic or heterocyclic intersugar linkages. Most preferred are oligonucleotides with phosphorothioate backbones and those with heteroatom backbones, particularly CH2-NH—O—CH2, CH,˜N(CH3)˜O˜CH2 (known as a methylene(methylimino) or MMI backbone], CH2-O—N(CH3)-CH2, CH2-N(CH3)-N(CH3)-CH2 and O—N(CH3)-CH2-CH2 backbones, wherein the native phosphodiester backbone is represented as O—P—O—CH,); amide backbones (see De Mesmaeker et al., Ace. Chem. Res. 28:366-374, 1995); morpholino backbone structures (see U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 254: 1497, 1991). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050 (each of which is incorporated by reference).

Morpholino-based oligomeric compounds are described in Braasch et al., Biochemistry 41(14):4503-4510, 2002; Genesis, volume 30, issue 3, 2001; Heasman, J., Dev. Biol., 243:209-214, 2002; Nasevicius et al., Nat. Genet. 26: 216-220, 2000; Lacerra et al., Proc. Natl. Acad. Sci. U.S.A. 97:9591-9596, 2000; and U.S. Pat. No. 5,034,506. Cyclohexenyl nucleic acid oligonucleotide mimetics are described in Wang et al., J. Am. Chem. Soc. 122, 8595-8602, 2000.

Modified oligonucleotide backbones that do not include a phosphorus atom therein have backbones that are formed by short-chain alkyl or cycloalkyl internucleoside linkages, mixed heteroatom and alkyl or cycloalkyl internucleoside linkages, or one or more short-chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439 (each of which is herein incorporated by reference).

One or more substituted sugar moieties can also be included, e.g., one of the following at the 2′ position: OH, SH, SCH₃, F, OCN, OCH₃ OCH₃, OCH₃O(CH₂)n CH₃, O(CH₂)n NH₂ or O(CH₂)n CH₃, where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-0-CH₂CH₂OCH₃, also known as 2′-O-(2-methoxyethyl)] (Martin et al., Helv. Chim. Acta 78:486, 1995). Other preferred modifications include 2′-methoxy (2′-0-CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics, such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC, and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, and 2,6-diaminopurine. See Kornberg, A., DNA Replication, W. H. Freeman & Co., San Francisco, 1980, pp 75-77; and Gebeyehu et al., Nucl. Acids Res. 15:4513, 1987. A “universal” base known in the art, e.g., inosine, can also be included. 5-Me-C substitutions have been shown to increase nucleic acid duplex stability by 0.6-1.2<0>C (Sanghvi, Y. S., in Crooke, S. T. and Lebleu, B., Eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science 254:1497-1500, 1991.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C), and uracil (U). Modified nucleobases comprise other synthetic and natural nucleobases, such as 5-methylcytosine (5-me-C), 5-hydroxymethyl cytosine, xanthine, hypoxanthine, 2-aminoadenine, 6-methyl, and other alkyl derivatives of adenine and guanine, 2-propyl and other alkyl derivatives of adenine and guanine, 2-thiouracil, 2-thiothymine, and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine, and 7-deazaadenine, and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., Ed. John Wiley & Sons, 1990, those disclosed by Englisch et al., Angewandle Chemie, International Edition′, 1991, 30, page 613, and those disclosed by Sanghvi, Y. S., Chapter 15, Antisense Research and Applications′, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines, and N-2, N-6 and O-6 substituted purines, comprising 2-aminopropyladenine, 5-propynyluracil, and 5-propynylcytosine. 5-methylcytosine substitutions have been shown to increase nucleic acid duplex stability by 0.6-1.2<0>C (Sanghvi, Y. S., Crooke, S. T. and Lebleu, B., Eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941 (each of which is herein incorporated by reference).

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety (Letsinger et al., Proc. Natl. Acad. Sci. U.S.A. 86:6553-6556, 1989), cholic acid (Manoharan et al., Bioorg. Med. Chem. Lett. 4:1053-1060, 1994), a thioether, e.g., hexyl-S-tritylthiol (Manoharan et al, Ann. N.Y. Acad. Sci. 660:306-309, 1992; Manoharan et al., Bioorg. Med. Chem. Lett. 3:2765-2770, 1993), a thiocholesterol (Oberhauser et al., Nucl. Acids Res. 20, 533-538, 1992), an aliphatic chain, e.g., dodecandiol or undecyl residues (Kabanov et al., FEBS Lett. 259:327-330, 1990; Svinarchuk et al., Biochimie 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett. 36:3651-3654, 1995; Shea et al., Nucl. Acids Res. 18:3777-3783, 1990), a polyamine or a polyethylene glycol chain (Mancharan et al., Nucleosides & Nucleotides 14:969-973, 1995), or adamantane acetic acid (Manoharan et al., Tetrahedron Lett. 36:3651-3654, 1995), a palmityl moiety (Mishra et al., Biochim. Biophys. Acta 1264: 229-237, 1995), or an octadecylamine or hexylamino-carbonyl-t oxycholesterol moiety (Crooke et al., J. Pharmacol. Exp. Ther. 277:923-937, 1996). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599,928 and 5,688,941 (each of which is herein incorporated by reference).

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism, or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941 (each of which is incorporated by reference).

The inhibitory nucleic acids useful in the present methods are sufficiently complementary to the target mRNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a mRNA, then the bases are considered to be complementary to each other at that position. In some embodiments, 100% complementarity is not required. In some embodiments, 100% complementarity is required. Routine methods can be used to design an inhibitory nucleic acid that binds to the target sequence with sufficient specificity.

While the specific sequences of certain exemplary target segments are set forth herein, one of skill in the art will recognize that these serve to illustrate and describe particular embodiments within the scope of the present invention. Additional target segments are readily identifiable by one having ordinary skill in the art in view of this disclosure. Target segments of 5, 6, 7, 8, 9, 10 or more nucleotides in length comprising a stretch of at least five (5) consecutive nucleotides within the seed sequence, or immediately adjacent thereto, are considered to be suitable for targeting as well. In some embodiments, target segments can include sequences that comprise at least the 5 consecutive nucleotides from the 5′-terminus of one of the seed sequence (the remaining nucleotides being a consecutive stretch of the same RNA beginning immediately upstream of the 5′-terminus of the seed sequence and continuing until the inhibitory nucleic acid contains about 5 to about 30 nucleotides). In some embodiments, target segments are represented by RNA sequences that comprise at least the 5 consecutive nucleotides from the 3 ‘-terminus of one of the seed sequence (the remaining nucleotides being a consecutive stretch of the same mRNA beginning immediately downstream of the 3’-terminus of the target segment and continuing until the inhibitory nucleic acid contains about 5 to about 30 nucleotides). One having skill in the art armed with the sequences provided herein will be able, without undue experimentation, to identify further preferred regions to target. In some embodiments, an inhibitory nucleic acid contain a sequence that is complementary to at least 5, 6, 7, 8, 9, 10, 11, 12, 13, 14, 15, 16, 17, 18, 19, 20, 21, 22, 23, 24, or 25 continguous nucleotides present in the target (e.g., one or both of ApoE and/or TREM2 mRNA).

Once one or more target regions, segments or sites have been identified, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this invention, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of an mRNA molecule, then the inhibitory nucleic acid and the mRNA are considered to be complementary to each other at that position. The inhibitory nucleic acids and the mRNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the inhibitory nucleic acid and the mRNA target. For example, if a base at one position of an inhibitory nucleic acid is capable of hydrogen bonding with a base at the corresponding position of an mRNA, then the bases are considered to be complementary to each other at that position. 100% complementarity is not required.

It is understood in the art that a complementary nucleic acid sequence need not be 100% complementary to that of its target nucleic acid to be specifically hybridizable. A complementary nucleic acid sequence for purposes of the present methods is specifically hybridisable when binding of the sequence to the target mRNA molecule interferes with the normal function of the target mRNA to cause a loss of expression or activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci. U.S.A. 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an mRNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol. 215:403-410, 1990; Zhang and Madden, Genome Res. 7:649-656, 1997). Antisense and other compounds of the invention that hybridize to an mRNA are identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

### Antisense

Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to the target mRNA, e.g., one or both of ApoE and/or TREM2. Thus, oligonucleotides are chosen that are sufficiently complementary to the target, i.e., that hybridize sufficiently well and with sufficient specificity, to give the desired effect.

### Modified Bases/Locked Nucleic Acids (LNAs)

In some embodiments, the inhibitory nucleic acids used in the methods described herein comprise one or more modified bonds or bases. Modified bases include phosphorothioate, methylphosphonate, peptide nucleic acids, or locked nucleic acid (LNA) molecules. Preferably, the modified nucleotides are locked nucleic acid molecules, including [alpha]-L-LNAs. LNAs comprise ribonucleic acid analogues wherein the ribose ring is “locked” by a methylene bridge between the 2′-oxygen and the 4′-carbon—i.e., oligonucleotides containing at least one LNA monomer, that is, one 2′-O,4′-C-methylene-β-D-ribofuranosyl nucleotide. LNA bases form standard Watson-Crick base pairs but the locked configuration increases the rate and stability of the base pairing reaction (Jepsen et al., Oligonucleotides 14:130-146, 2004). LNAs also have increased affinity to base pair with RNA as compared to DNA. These properties render LNAs especially useful as probes for fluorescence in situ hybridization (FISH) and comparative genomic hybridization, as knockdown tools for mRNAs, and as antisense oligonucleotides to target mRNAs.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60, 2006; McTigue et al., Biochemistry 43:5388-405, 2004; and Levin et al., Nucl. Acids. Res. 34:e142, 2006. For example, “gene walk” methods, similar to those used to design antisense oligos, can be used to optimize the inhibitory activity of the LNA; for example, a series of oligonucleotides of 10-30 nucleotides spanning the length of a target mRNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of three or more Gs or Cs, or more than four LNA residues, should be avoided where possible (for example, it may not be possible with very short (e.g., about 9-10 nt) oligonucleotides). In some embodiments, the LNAs are xylo-LNAs.

In some embodiments, the LNA molecules can be designed to target a specific region of the mRNA. For example, a specific functional region can be targeted, e.g., a region comprising a seed sequence. Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol. 215:403-410, 1990; Zhang and Madden, Genome Res. 7:649-656, 1997), e.g., using the default parameters.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 2010/0267018; 2010/0261175; and 2010/0035968; Koshkin et al., Tetrahedron 54:3607-3630, 1998; Obika et al., Tetrahedron Lett. 39:5401-5404, 1998; Jepsen et al., Oligonucleotides 14:130-146, 2004; Kauppinen et al., Drug Disc. Today 2(3):287-290, 2005; and Ponting et al., Cell 136(4):629-641, 2009, and references cited therein.

See also U.S. Ser. No. 61/412,862, which is incorporated by reference herein in its entirety.

siRNA

In some embodiments, the nucleic acid sequence that is complementary to a target mRNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, 2002; Lee et al., Nature Biotechnol., 20, 500-505, 2002; Miyagishi and Taira, Nature Biotechnol. 20:497-500, 2002; Paddison et al., Genes & Dev. 16:948-958, 2002; Paul, Nature Biotechnol. 20, 505-508, 2002; Sui, Proc. Natl. Acad. Sci. U.S.A., 99(6):5515-5520, 2002; Yu et al., Proc. Natl. Acad. Sci. U.S.A. 99:6047-6052, 2002.

The target RNA cleavage reaction guided by siRNAs is highly sequence specific. In general, siRNA containing a nucleotide sequences identical to a portion of the target nucleic acid (i.e., a target region comprising the seed sequence of a target mRNA) are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

### Ribozymes

Trans-cleaving enzymatic nucleic acid molecules can also be used; they have shown promise as therapeutic agents for human disease (Usman & McSwiggen, Ann. Rep. Med. Chem. 30:285-294, 1995; Christoffersen and Marr, J. Med. Chem. 38:2023-2037, 1995). Enzymatic nucleic acid molecules can be designed to cleave specific mRNA targets within the background of cellular RNA. Such a cleavage event renders the mRNA non-functional.

In general, enzymatic nucleic acids with RNA cleaving activity act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its activity. After an enzymatic nucleic acid has bound and cleaved its RNA target, it is released from that RNA to search for another target and can repeatedly bind and cleave new targets.

Several approaches such as in vitro selection (evolution) strategies (Orgel, Proc. R. Soc. London, B 205:435, 1979) have been used to evolve new nucleic acid catalysts capable of catalyzing a variety of reactions, such as cleavage and ligation of phosphodiester linkages and amide linkages, (Joyce, Gene, 82, 83-87, 1989; Beaudry et al., Science 257, 635-641, 1992; Joyce, Scientific American 267, 90-97, 1992; Breaker et al., TIBTECH 12:268, 1994; Bartel et al., Science 261:1411-1418, 1993; Szostak, TIBS 17, 89-93, 1993; Kumar et al., FASEB J., 9:1183, 1995; Breaker, Curr. Op. Biotech., 1:442, 1996). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 rnM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

### Sense Nucleic Acids—Genetic Therapy

Agents useful in the methods of treatment described herein include sense nucleic acid molecules that increase the expression or activity of Mertk or Egr1, e.g., nucleic acid molecules that comprise sequences encoding a functional human Mertk or Egr1 protein. A sense nucleic acid can be contain a sequence that is at least 80% (e.g., at least 90%, 95%, 96%, 97%, 98%, 99%, or 100%) identical to the reference sequences provided herein, e.g., to the full length of the reference sequence, optionally without any signal sequence. Sense nucleic acids can contain one or more of any of the modifications (e.g., backbone modifications, nucleobase modifications, sugar modifications, or one or more conjugated molecules) described herein without limitation. Methods of making and administering sense nucleic acids are known in the art. Additional methods of making and using sense nucleic acids are described herein.

The sense nucleic acids described herein, e.g., nucleic acids encoding an Mertk and/or Egr1 polypeptide or active fragment thereof, or a nucleic acid encoding a protein that increases Mertk and/or Egr1 expression, level or activity, can be incorporated into a gene construct to be used as a part of a gene therapy protocol. The invention includes targeted expression vectors for in vivo transfection and expression of a polynucleotide that encodes an Mertk and/or Egr1 polypeptide or active fragment thereof, or a protein that increases Mertk and/or Egr1 expression, level, or activity as described herein, in particular cell types, especially microglial cells. Expression constructs of such components can be administered in any effective carrier, e.g., any formulation or composition capable of effectively delivering the component gene to cells in vivo. Approaches include insertion of the gene in viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (lipofectamine) or derivatized (e.g., antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol 158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). In some embodiments, the AAV is an ancestral or synthetic viral vector, e.g., as described in Zinn et al., 12(6):1056-1068, 2015.

In addition to viral transfer methods, such as those illustrated above, non-viral methods can also be employed to cause expression of a nucleic acid compound described herein (e.g., a Mertk and/or Egr1 nucleic acid or a nucleic acid encoding a compound that increases Mertk and/or Egr1 expression, levels or activity) in the tissue of a subject. Typically non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol. 116(1):131-135 (2001); Cohen et al., Gene Ther. 7(22):1896-905 (2000); or Tam et al., Gene Ther. 7(21):1867-74 (2000).

In some embodiments, a sense nucleic acid encoding a Mertk and/or Egr1 is entrapped in liposomes bearing positive charges on their surface (e.g., lipofectins), which can be tagged with antibodies against cell surface antigens of the target tissue (Mizuno et al., No Shinkei Geka 20:547-551 (1992); PCT publication WO91/06309; Japanese patent application 1047381; and European patent publication EP-A-43075).

In clinical settings, the gene delivery systems for the therapeutic nucleic acid can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection (e.g., Chen et al., PNAS USA 91: 3054-3057 (1994)).

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

### Making and Using Inhibitory Nucleic Acids and Sense Nucleic Acids

The nucleic acid sequences used to practice the methods described herein, whether inhibitory DNA or RNA, mRNA, cDNA, genomic DNA, vectors, viruses or hybrids thereof, can be isolated from a variety of sources, genetically engineered, amplified, synthesized and/or expressed/generated recombinantly. Recombinant nucleic acid sequences can be individually isolated or cloned and tested for a desired activity. Any recombinant expression system can be used, including e.g., in vitro, bacterial, fungal, mammalian, yeast, insect, or plant cell expression systems.

Nucleic acid sequences of the invention (e.g., any of the inhibitory nucleic acids or sense nucleic acids described herein) can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)).

As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, herpes virus, adenovirus, adeno-associated virus, pox virus, or alphavirus. The recombinant vectors (e.g., viral vectors) capable of expressing the nucleic acids of the invention can be delivered as described herein, and persist in target cells (e.g., stable transformants). For example, such recombinant vectors (e.g., a recombinant vector that results in the expression of an antisense oligomer that is complementary to hsa-miR-155) can be administered into (e.g., injection or infusion into) the cerebrospinal fluid of the subject (e.g., intracranial injection, intraparenchymal injection, intraventricular injection, and intrathecal injection, see, e.g., Bergen et al., Pharmaceutical Res. 25:983-998, 2007). A number of exemplary recombinant viral vectors that can be used to express any of the nucleic acids described herein are also described in Bergen et al. (supra). Additional examples of recombinant viral vectors are known in the art.

The nucleic acids provided herein (e.g., the inhibitory nucleic acids) can be further be complexed with one or more cationic polymers (e.g., poly-L-lysine and poly(ethylenimine), cationic lipids (e.g., 1,2-dioleoyl-3-trimethylammonium propone (DOTAP), N-methyl-4-(dioleyl)methylpyridinium, and 313-[N—(N′,N′-dimethylaminoethane)-carbamoyl] cholesterol), and/or nanoparticles (e.g., cationic polybutyl cyanoacrylate nanoparticles, silica nanoparticles, or polyethylene glycol-based nanoparticles) prior to administration to the subject (e.g., injection or infusion into the cerebrospinal fluid of the subject). Additional examples of cationic polymers, cationic lipids, and nanoparticles for the therapeutic delivery of nucleic acids are known in the art. The therapeutic delivery of nucleic acids has also been shown to be achieved following intrathecal injection of polyethyleneimine/DNA complexes (Wang et al., Mol. Ther. 12:314-320, 2005). The methods for delivery of nucleic acids described herein are non-limiting. Additional methods for the therapeutic delivery of nucleic acids to a subject are known in the art.

In some embodiments, the inhibitory nucleic acids (e.g., one or more inhibitory nucleic acids targeting one or both of ApoE and/or TREM2) can be administered systemically (e.g., intravenously, intaarterially, intramuscularly, subcutaneously, or intraperitoneally) or intrathecally (e.g., epidural administration). In some embodiments, the inhibitory nucleic acid is administered in a composition (e.g., complexed with) one or more cationic lipids. Non-limiting examples of cationic lipids that can be used to administer one or more inhibitory nucleic acids (e.g., any of the inhibitory nucleic acids described herein) include: Lipofectamine, the cationic lipid molecules described in WO 97/045069, and U.S. Patent Application Publication Nos. 2012/0021044, 2012/0015865, 2011/0305769, 2011/0262527, 2011/0229581, 2010/0305198, 2010/0203112, and 2010/0104629 (each of which is herein incorporated by reference). Nucleic acid sequences used to practice this invention can be synthesized in vitro by well-known chemical synthesis techniques, as described in, e.g., Adams, J. Am. Chem. Soc. 105:661, 1983; Belousov, Nucleic Acids Res. 25:3440-3444, 1997; Frenkel, Free Radic. Biol. Med. 19:373-380, 1995; Blommers, Biochemistry 33:7886-7896, 1994; Narang, Meth. Enzymol. 68:90, 1994; Brown, Meth. Enzymol. 68:109, 1979; Beaucage, Tetra. Lett. 22:1859, 1981; and U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2(3):287-290, 2005; Koshkin et al., J. Am. Chem. Soc., 120(50):13252-13253, 1998). For additional modifications see US 2010/0004320, US 2009/0298916, and US 2009/0143326 (each of which is incorporated by reference).

Techniques for the manipulation of nucleic acids used to practice this invention, such as, e.g., subcloning, labeling probes (e.g., random-primer labeling using Klenow polymerase, nick translation, amplification), sequencing, hybridization, and the like are well described in the scientific and patent literature, see, e.g., Sambrook et al., Molecular Cloning; A Laboratory Manual 3d ed. (2001); Current Protocols in Molecular Biology, Ausubel et al., Eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, Ed. Elsevier, N.Y. (1993).

### Antibodies and Recombinant Proteins

One or more antibodies that specifically bind to a ApoE or TREM2 protein can also be administered to a subject to treat a neurodegenerative disease. Antibodies that specifically bind to APOE or TREM2 proteins are either commercially available (e.g., to APOE from AB947 (Millipore), NB110-60531 (Novus Biologicals), LS-B6780/43356 (Lifespan Bioscience) and EP1373Y (Epitomics) and to TREM2 from (R&D Systems)) or can be generated using standard methods known in the art. For example, a polyclonal antibody that specifically binds to ApoE or TREM2 can be generated by immunizing a mammal with the purified protein and isolating antibodies from the mammal that specifically bind to the purified protein. The antibodies used can be a monoclonal or polyclonal antibody. The antibodies administered can be a immunoglobulin G or immunoglobulin M. The antibodies administered can be chimeric (e.g., a humanized antibody) or a human antibody. The antibodies used can also be an antibody fragment (e.g., a Fab, F(ab′)₂, Fv, and single chain Fv (scFv) fragment).

In some embodiments, APOE inhibitors for use in the present invention are anti-APOE antibodies, such as AB947 (Millipore), NB110-60531 (Novus Biologicals), LS-B6780/43356 (Lifespan Bioscience) and EP1373Y (Epitomics).

In some embodiments, APOE4-specific antibodies for use in the present invention preferably include those commercially available from Bio Vision, MBL International, Covance, or IBL (American lmmuno-Biological Laboratories).

Further, in a particular embodiment of the present invention, the APOE inhibitor is a soluble receptor for LDL (LDLR), such as the recombinant human LDL R 2148-LD/CF (R&D SYSTEMS).

### Pharmaceutical Compositions

The methods described herein can include the administration of pharmaceutical compositions and formulations comprising any of the inhibitory nucleic acids (e.g., one or more inhibitory nucleic acids targeting Trem2 and/or ApoE), sense nucleic acids encoding Egr1 and/or Mertk, peptides, small molecules, or antibodies described herein that bind to and inhibit Trem2 and/or ApoE.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the compositions. In some embodiments, one or more cationic lipids, cationic polymers, or nanoparticles can be included in compositions containing the one or more inhibitory nucleic acids (e.g., compositions containing one or more inhibitory nucleic acids targeting Trem2 and/or ApoE).

Formulations of the compositions of the invention include those suitable for intradermal, inhalation, oral/nasal, topical, parenteral, intrathecal or other route of administration as known in the art or described herein. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect.

Pharmaceutical formulations of this invention can be prepared according to any method known to the art for the manufacture of pharmaceuticals. Such drugs can contain sweetening agents, flavoring agents, coloring agents, and preserving agents. A formulation can be admixtured with nontoxic pharmaceutically acceptable excipients which are suitable for manufacture. Formulations may comprise one or more diluents, emulsifiers, preservatives, buffers, excipients, etc., and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., inhibitory nucleic acids or sense nucleic acids described herein) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth, and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long-chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents, and one or more sweetening agents, such as sucrose, aspartame, or saccharin. Formulations can be adjusted for osmolarity.

In some embodiments, oil-based pharmaceuticals are used for administration of nucleic acid sequences of the invention. Oil-based suspensions can be formulated by suspending an active agent in a vegetable oil, such as arachis oil, olive oil, sesame oil, or coconut oil, or in a mineral oil such as liquid paraffin; or a mixture of these. See e.g., U.S. Pat. No. 5,716,928, describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin, or cetyl alcohol. Sweetening agents can be added to provide a palatable oral preparation, such as glycerol, sorbitol, or sucrose. These formulations can be preserved by the addition of an antioxidant such as ascorbic acid. As an example of an injectable oil vehicle, see Minto, J. Pharmacol. Exp. Ther. 281:93-102, 1997.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters, or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate, and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi, J. Clin. Pharmacol. 35:1187-1193, 1995; Tjwa, Ann. Allergy Asthma Immunol. 75:107-111, 1995). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can also be delivered as microspheres for slow release in the body. For example, microspheres can be administered via intradermal injection of drug which slowly release subcutaneously; see Rao, J. Biomater Sci. Polym. Ed. 7:623-645, 1995; as biodegradable and injectable gel formulations, see, e.g., Gao, Pharm. Res. 12:857-863, 1995; or, as microspheres for oral administration, see, e.g., Eyles, J. Pharm. Pharmacol. 49:669-674, 1997.

In some embodiments, the pharmaceutical compounds can be parenterally administered, such as by intravenous (IV) administration or administration into a body cavity, a lumen of an organ, or into the cranium (e.g., intracranial injection or infusion) or the cerebrospinal fluid of a subject. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids, such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate, and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid or a sense nucleic acid can be made by lyophilizing a solution comprising a pharmaceutical of the invention and a bulking agent, e g, mannitol, trehalose, raffinose, and sucrose, or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5, but less than 6.5. See, e.g., US2004/0028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989.

The formulations of the invention can be administered for prophylactic and/or therapeutic treatments. In some embodiments, for therapeutic applications, compositions are administered to a subject who is at risk of or has a disorder described herein, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to reduce the number of symptoms or reduce the severity, duration, or frequency of one or more symptoms of a neurodegenerative disorder in a subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age, and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones, J. Steroid Biochem. Mol. Biol. 58:611-617, 1996; Groning, Pharmazie 51:337-341, 1996; Fotherby, Contraception 54:59-69, 1996; Johnson, J. Pharm. Sci. 84:1144-1146, 1995; Rohatagi, Pharmazie 50:610-613, 1995; Brophy, Eur. J. Clin. Pharmacol. 24:103-108, 1983; Remington: The Science and Practice of Pharmacy, 21st ed., 2005). The state of the art allows the clinician to determine the dosage regimen for each individual patient, active agent, and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, and the like. The formulations should provide a sufficient quantity of active agent to effectively treat, prevent or ameliorate conditions, diseases, or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray, or inhalation. Actual methods for preparing parenterally or non-parenterally administrable formulations will be known or apparent to those skilled in the art and are described in more detail in such publications as Remington: The Science and Practice of Pharmacy, 21st ed., 2005.

Various studies have reported successful mammalian dosing using complementary nucleic acid sequences. For example, Esau C., et al., Cell Metabolism, 3(2):87-98, 2006, reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST 3/4 45, ALT 3/4 35) for all doses with the exception of the 75 mg/kg dose of miR-122 ASO, which showed a very mild increase in ALT and AST levels. They concluded that 50 mg/kg was an effective, non-toxic dose. Another study by Krützfeldt J., et al., Nature 438, 685-689, 2005, injected anatgomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen et al., Nature 452, 896-899, 2008, report that efficient silencing of miR-122 was achieved in primates by three doses of 10 mg kg-1 LNA-antimiR, leading to a long-lasting and reversible decrease in total plasma cholesterol without any evidence for LNA-associated toxicities or histopathological changes in the study animals.

In some embodiments, the methods described herein can include co-administration with other drugs or pharmaceuticals, e.g., any of the treatments of a neurodegenerative disorder described herein.

### Examples

The invention is further described in the following examples, which do not limit the scope of the invention described in the claims.

Materials and Methods

The following materials and methods were used in the Examples below.

Mice.

C57BL6 females, APOE−/−, Egr1−/−, Mertk−/−, Axl−/− and miR155−/− mice were obtained from Jaxmice laboratories. Trem2−/− mice were provided by Dr. Christian Haass (Munich, Germany. All mice were housed with food and water ad libitum. Mice were killed by CO₂ inhalation. The Institutional Animal Care and Use Committee at Harvard Medical School approved all experimental procedures involving animals.

Induction of EAE.

EAE was induced by immunization of female C57B16 mice with MOG₃₅₋₅₅ peptide emulsified in CFA (100 μg per mouse), followed by the administration of pertussis toxin (150 ng per mouse) at day 0 and 2. Clinical signs of EAE were assessed according to the following score: 0, no signs of disease; 1, loss of tone in the tail; 2, hind limb paresis; 3, hind limb paralysis; 4, tetraplegia; 5, moribund. the duration of the acute phase (15 d), or only during the progressive or chronic phase (days 30-50).

Mouse Microglia Isolation and Sorting.

Microglia isolation was done according to previously described (Butovsky et al, 2014). Briefly, mice were transcardially perfused with ice-cold phosphate-buffer saline (PBS), spinal cords and brains separately dissected. Single cell suspensions were prepared and centrifuged over a 37%/70% discontinuous Percoll gradient (GE Healthcare), mononuclear cells isolated from the interface. Isolated cells were labeled with combination of anti-FCRLS (monoclonal antibody, clone 4G11, Butovsky et al., 2014), followed by secondary detection with goat anti-rat IgG conjugated to APC (clone Poly4054, Biolegend) and then anti-CD11b (CD11b-PeCy7 clone M1/70, BD Biosciences) antibodies to specifically sort resident microglia.

Phagocytic versus non phagocytic microglia were further sorted from the FCRLS⁺/CD11b⁺-population by detection of Alexa488 fluorescence.

Adult Mouse Microglial Culture and Generation of M0, M1 and M2 Cultures.

Adult microglia were isolated from C57BL/6 mice at age 6-10 weeks from brains as described recently with slight modifications (NN). Briefly, microglia were isolated and sorted as described above, cultured in 96-well plate (2×10⁴ cells per well in 200μl) in poly-D-lysine-coated plates (BD Biosciences), and grown in microglia culture medium (DMEM/F-12 Glutamax; Invitrogen) with 10% FCS, 100 U ml⁻¹ penicillin and 100 mg ml⁻¹ streptomycin at 37° C., 5% CO₂ supplemented with the following: To generate M0 microglia, mouse recombinant carrier-free MCSF 10 ng ml⁻¹ (R&D Systems) and 50 ng ml⁻¹ human recombinant TGFβ1 (Miltenyi Biotec) was added to microglia culture medium. M1 and M2 microglia were polarized as described elsewhere (7). Cells were cultured for at least 5 days without changing media before treatment with additional substances.

Isolation of Primary Neurons.

Primary neurons were prepared from embryos at age E18.5. Cerebral hemispheres were isolated and freed from meninges. Tissues were digested with 0.25% trypsin in HBSS for 15 min at 37° C., then washed three times with HBSS and triturated with fire-polished glass pipettes to obtain single cells. This cell suspension was filtered through a 70- and a 40-μm cell strainer and subjected to a spin at 1,000 g for 5 minutes. Cell pellet was resuspended with fresh 10 ml of HBSS. Cell density was then determined with a hemocytometer and cells were seeded at different densities according to the experimental design and need. We used DMEM supplemented with 10% FBS for the initial plating, and the medium was changed to Neurobasal supplemented with 1×B27 (Invitrogen) in 3 h. Half of the medium was changed every 3 d.

Induction of Cell Death

Neurons were carefully detached from the plate surface by repeated washes with PBS. Subsequently, neurons were irradiated with UV light for 15 minutes. Afterwards, cells were harvested by centrifugation and the pellet processed for downstream applications.

Labeling of Neurons

Apoptotic neurons were resuspended in 1 ml PBS and incubated for 15 minutes at 37° C. with 1-2 μl of the labeling dye (Alexa488 5-SDP Ester; #A30052 life technologies; dissolve one vial of A30052 (1 mg) in 100 μl anhydrous DMSO). To stop the reaction, cells were washed in PBS and harvested by centrifugation for 7 minutes at 1200 rpm. To block and capture residual dye, cells were resuspended in 1 ml pure FBS and washed with PBS. Neurons were harvested by centrifugation, the pellet was resuspended in PBS, harvested again and resuspended in 1 ml PBS. Total cell number was determined with a Neubauer counting chamber, cells were harvested again and resuspended in the final volume of PBS at a density of approximately 100.000 ells/4 μl for stereotactic injection.

RNA Isolation, Quantitative Real-Time PCR, Nanostring RNA Counting.

Total RNA was extracted using mirVana™ miRNA isolation kit (Ambion) according to the manufacturer's protocol.

Total RNA (20-40 ng) was used in 20-40 μl of reverse transcription reaction according to the manufacturer (high-capacity cDNA Reverse Transcription Kit; Applied Biosystems) and 3 ng of RNA in 5 μl reverse transcription reaction with specific miRNA probes (Applied Biosystems). mRNA or miRNAs levels were normalized relative to U6 or GAPDH, respectively, by the formula 2^{-ΔCt}, where ΔCt=Ct_{miR-X}−Ct_{U6} or GAPDH. Real-time PCR reaction was performed using Vii? (Applied Biosystems). All qRT-PCRs were performed in duplicate or triplicate, and the data are presented as relative expression compared to GAPDH or U6 as mean±s.e.m.

Nanostring nCounter technology allows expression analysis of multiple genes from a single sample. We performed nCounter multiplexed target profiling of 179 inflammation genes which consist of genes differentially expressed during inflammation and immune responses, nCounter 578 miRNA (see complete list of genes and miRNAs at and 400 microglial transcripts (MG400, see MG400 chip design). 100 ng per sample of total RNA were used in all described nCounter analyses according to the manufacturer's suggested protocol.

Human Brain Specimens.

Fresh human brain was obtained from Massachusetts General Hospital pathology department within 5 h of time of death or immersion fixated in 4% PFA. Tissue was used for immunohistochemical analysis or Laser capturing.

IPA (Ingenuity) Analysis.

Data were analyzed by IPA (Ingenuity Systems). Differentially expressed genes (with corresponding fold changes and P values) were incorporated in canonical pathways and bio-functions and were used to generate biological networks. Uploaded data set for analysis were filtered using the following cutoff definitions: fivefold change, P<0.01. IPA provides the most comprehensive, validated knowledgebase of interactions between biomolecules including miRNA. Furthermore, they also provide comprehensive annotation of different functional and pathway enrichment along with the ability to present this knowledge in the form of a network of interaction.

MG400, MG447 and MG550 Chip Design.

The MG400 chip was designed using the quantitative NanoString nCounter platform as described previously (7).

The MG447 chip was designed using the quantitative NanoString nCounter platform based on the MG400 chip. The chip includes 447 genes related to: (1) 376 microglial genes; (2) 40 inflammation genes that we found affected in microglia in mouse models of SOD1, EAE, and Alzheimer's disease; (3) 25 known/predicted miR-155 targeted genes; and (4) 6 housekeeping genes.

The MG400 chip was designed using the quantitative NanoString nCounter platform. Selection of genes was based on analyses that identified genes and proteins which are specifically or highly expressed in adult mouse microglia plus 40 inflammation-related genes which were significantly affected in EAE, APP/PS1 and SOD1 mice (NN). In addition, MG468 contains. MG550 includes an additional set of affected genes identified in AD, ALS, and EAE mouse models.

Immunohistochemistry.

Following immersion fixation in 4% PFA, brains were dehydrated and embedded in paraffin. Frontal sections (2-5μm) were collected on superfrost slides, deparaffinazied in xylole and rehydrated.

For immunofluorescence stainings, antigen retrieval was performed for 30 min at 96° C. in 10 mM citrate buffer pH 6.0. Subsequently, sections were permeabilized with 0.2% TritonX-100 (Roche) in TBS. Tissues were washed, blocked in Pierce Protein-Free T20 (TBS) blocking buffer (#37571, Thermo Scientific) and treated with 1% Sudan Black to reduce autofluorescence. Sections were stained with polyclonal rabbit antibody to P2ry12 (1:300 in blocking buffer) at 4° C. overnight, washed with TBS-T and incubated with secondary donkey anti-rabbit Alexa555 labeled antibody (Life Technologies; 1:300 in blocking buffer) for 90 minutes. Sections were washed again and slides were mounted with DAPI-Fluoromount-G (SouthernBiotech, Birmingham, USA). Data acquisition was performed using a Leica TCS SP5 confocal microscope and Leica application suite software (LAS-AF-lite).

Statistical Analysis.

For all statistical analyses, data distribution was assumed to be normal, but this was not formally tested. Unless otherwise indicated, data are presented as mean±s.e.m. and two-tailed Student's t tests (unpaired) or ANOVA multiple comparison tests were used to assess statistical significance and calculated with GraphPad Prism 6 software. No statistical methods were used to predetermine sample sizes, but our sample sizes are similar to those reported in previous publication Butovsky et al., J. Clin. Invest. 122, 3063-3087 (2012). Data collection and analysis were performed blind to the conditions of the experiments. Also, data for each experiment were collected and processed randomly and animals were assigned to various experimental groups randomly as well. All n and P values and statistical tests are indicated in the figure legends.

### Example 1. Reciprocal Dysregulation of APOE and TGFbeta1 Signaling is a Common Pathway in Disease-Associated Microglia

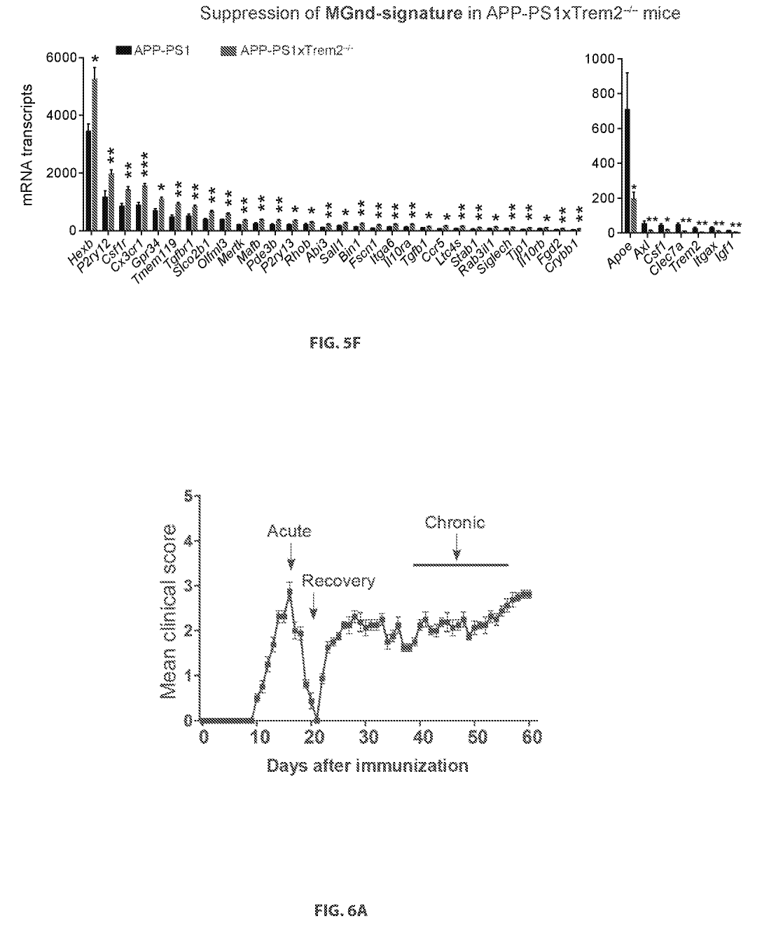

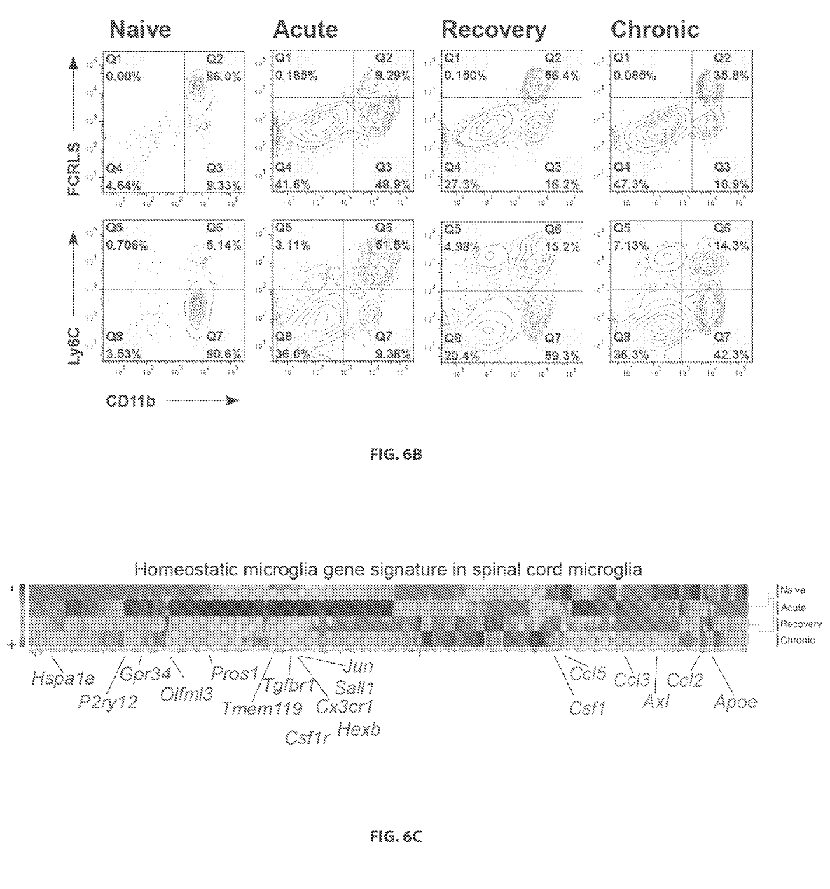

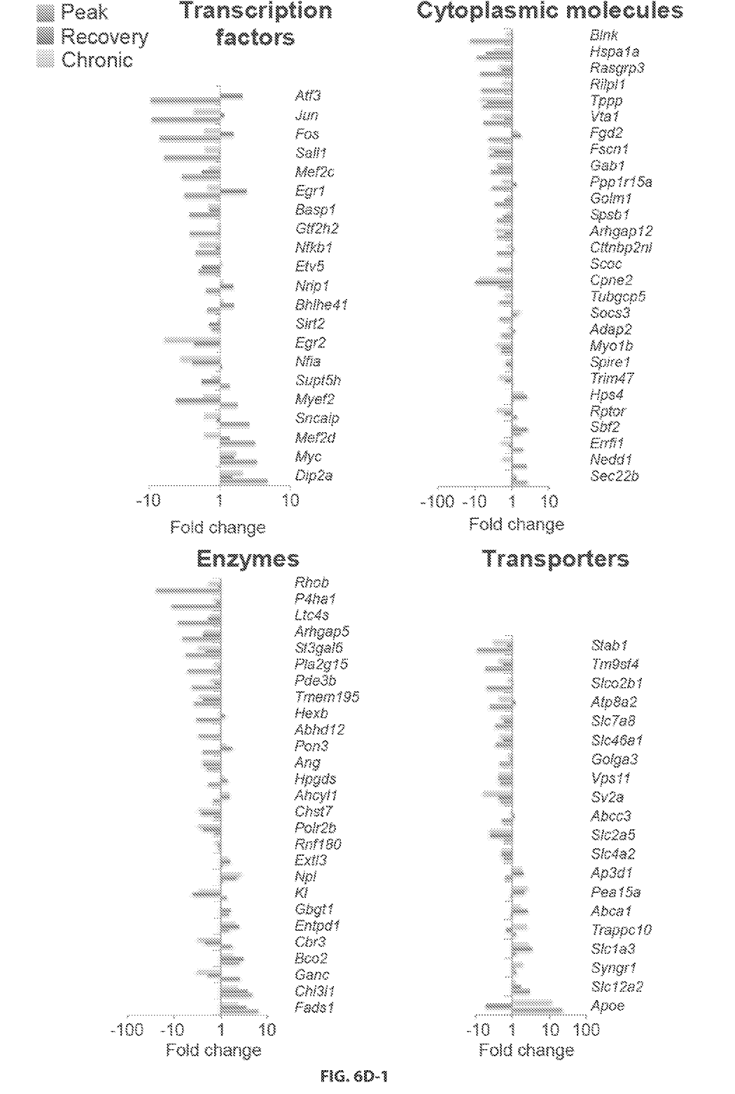

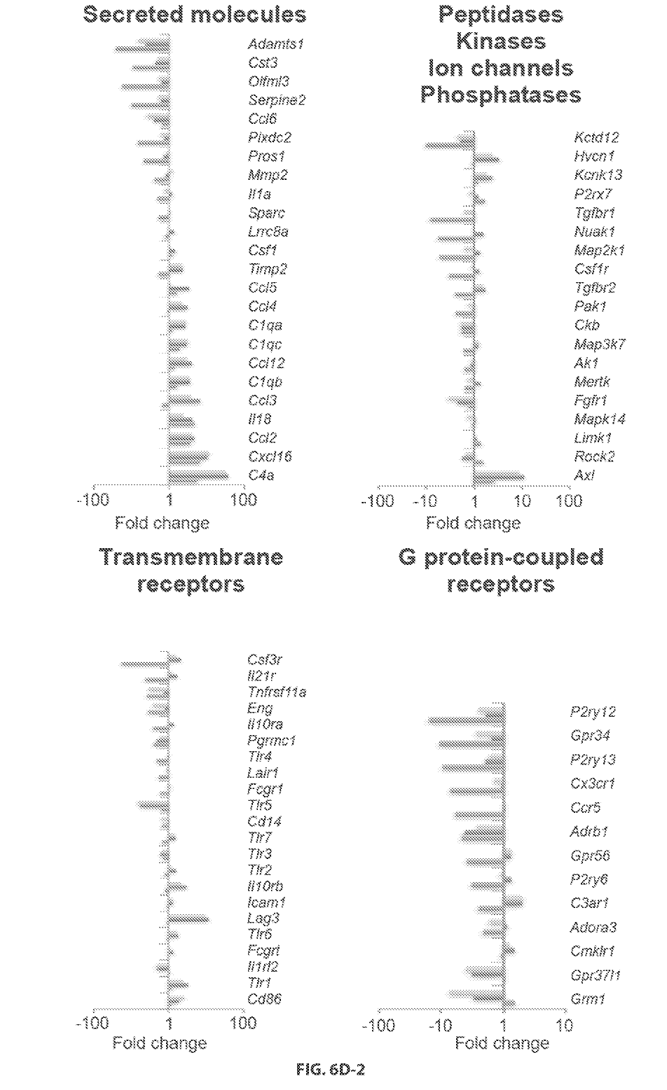

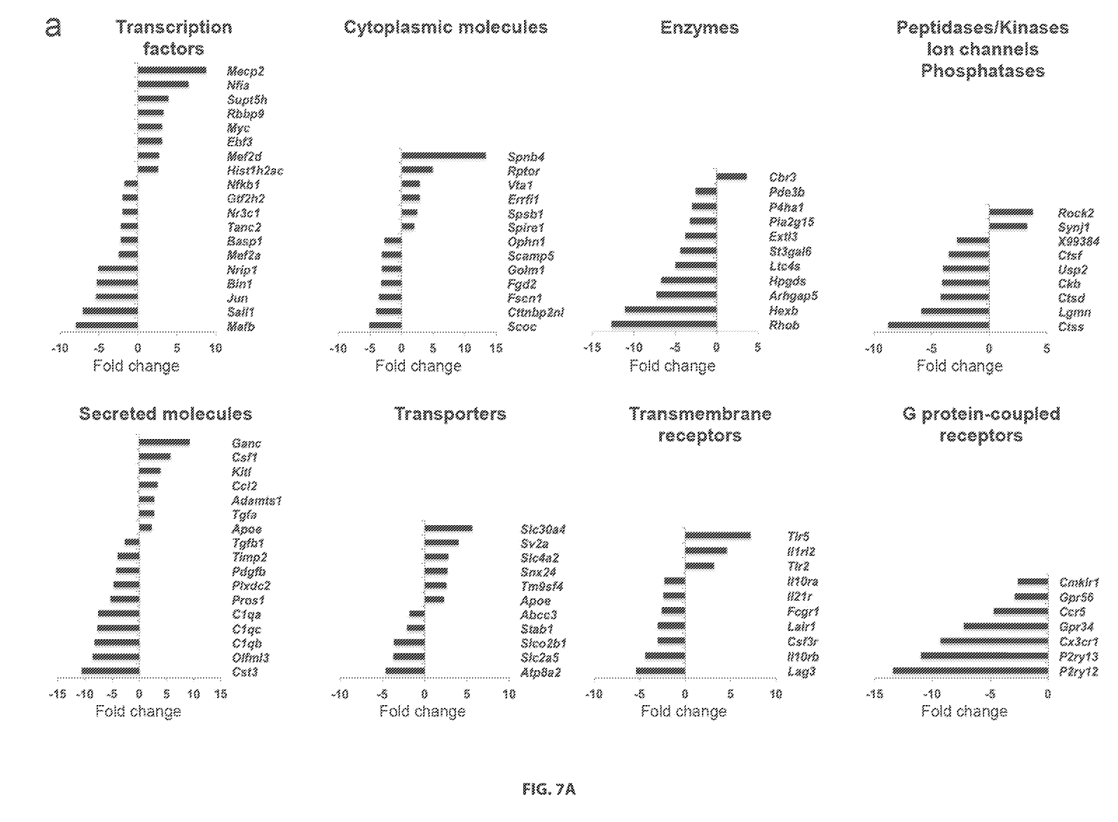

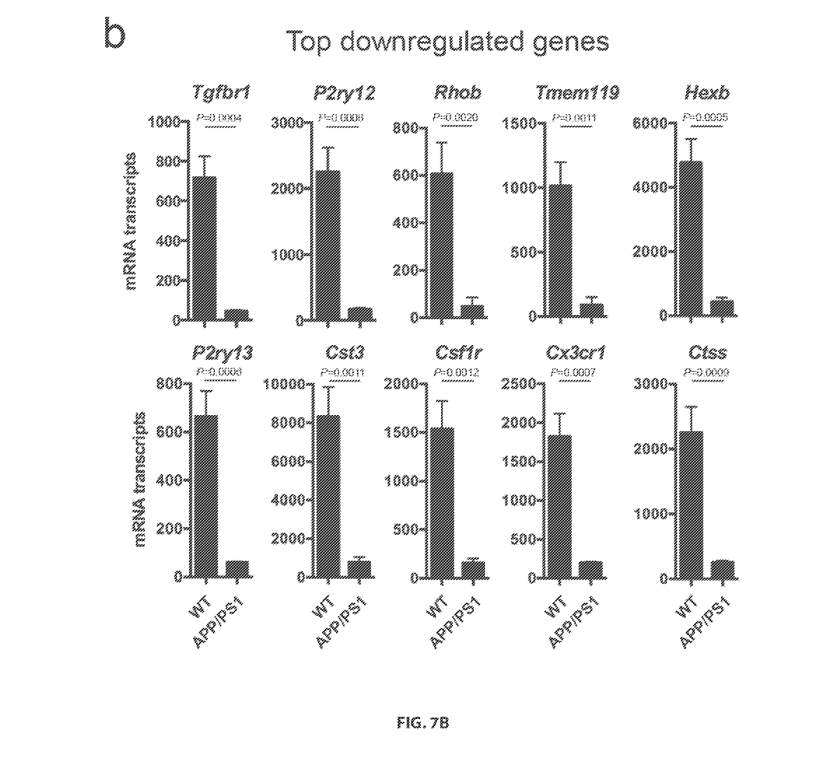

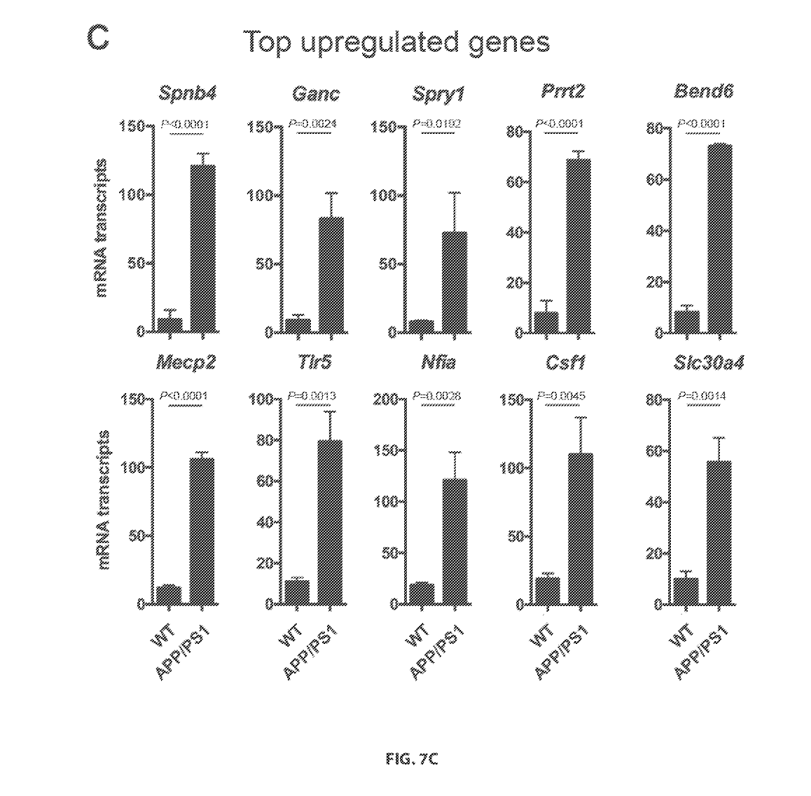

The present inventors characterized the molecular signature of homeostatic microglia in the healthy brain using microglia specific antibodies (7). To better understand what is happening in the diseased brain, we intensively profiled microglia by Nanostring gene analysis in different diseases stages of neurodegenerative (AD) or neuroinflammatory (ALS, MS) conditions in mouse models (ALS (SODG93A); AD (APP/PS1); MS: experimental autoimmune encephalomyelitis (EAE)) using our custom MG400 chip that contains unique and enriched microglial genes, and Nanostring mouse inflammation chip (described in ref 7). In EAE, sorting of spinal cord microglia was performed at different disease stages (FIGS. 6A-D, EAE microglia signature) and for all sorting, we used microglia specific antibody FCRLS (FCRLS⁺/CD11b⁺/Ly6C⁻) (7). Interestingly, the homeostatic signature was suppressed in all three investigated diseases, although to different degree (FIGS. 6c and d: EAE microglia signature; FIGS. 7a-c: AD microglia signature; FIG. 8: SOD1 microglia signature). Of note, the global metabolism of microglia was severely suppressed in clinical SOD1-mice with downregulation of almost all microglia specific genes (FIG. 8).

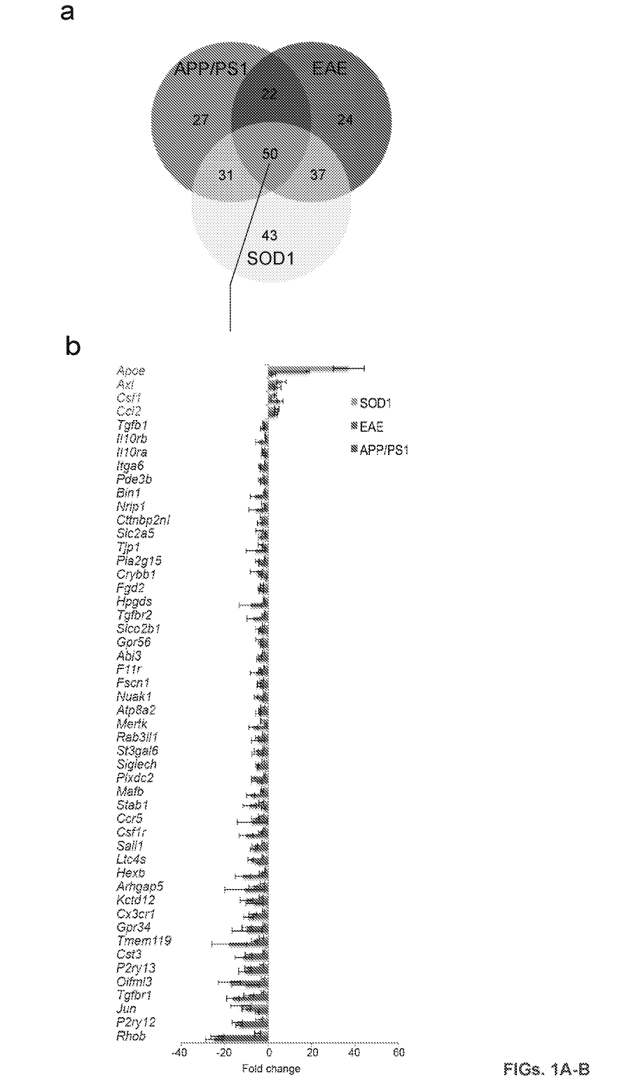

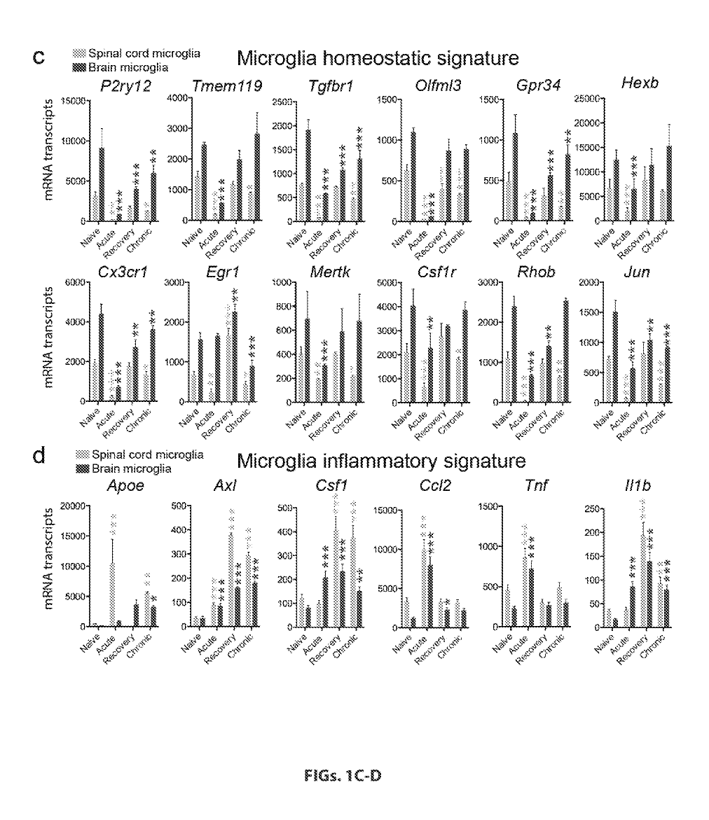

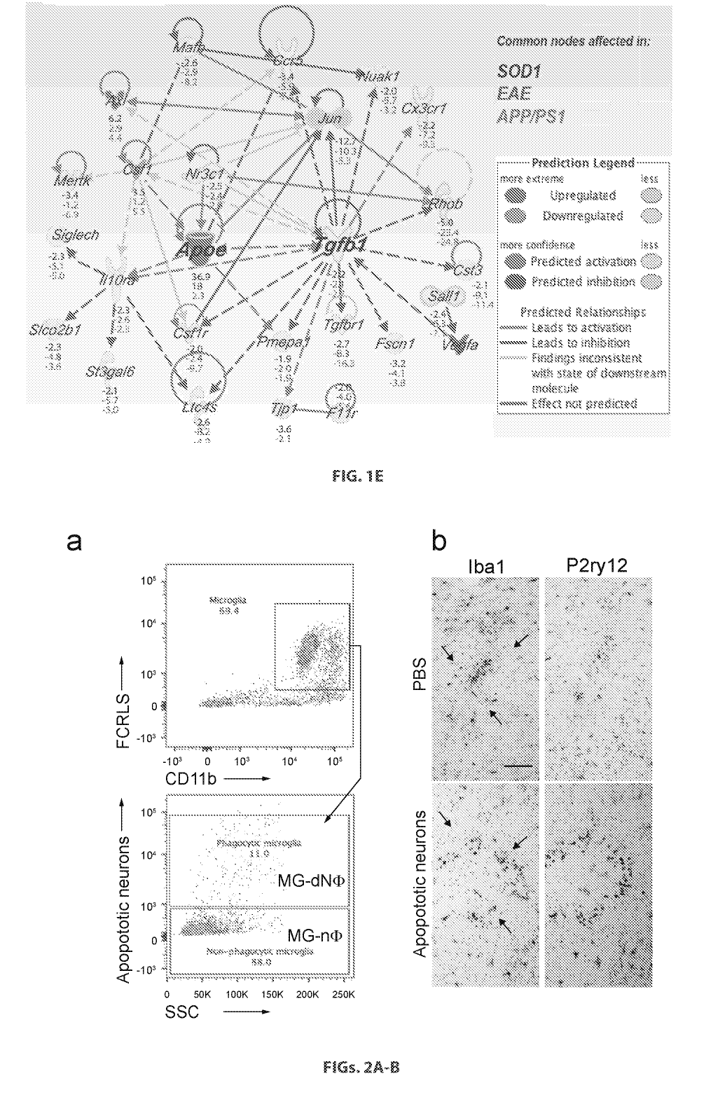

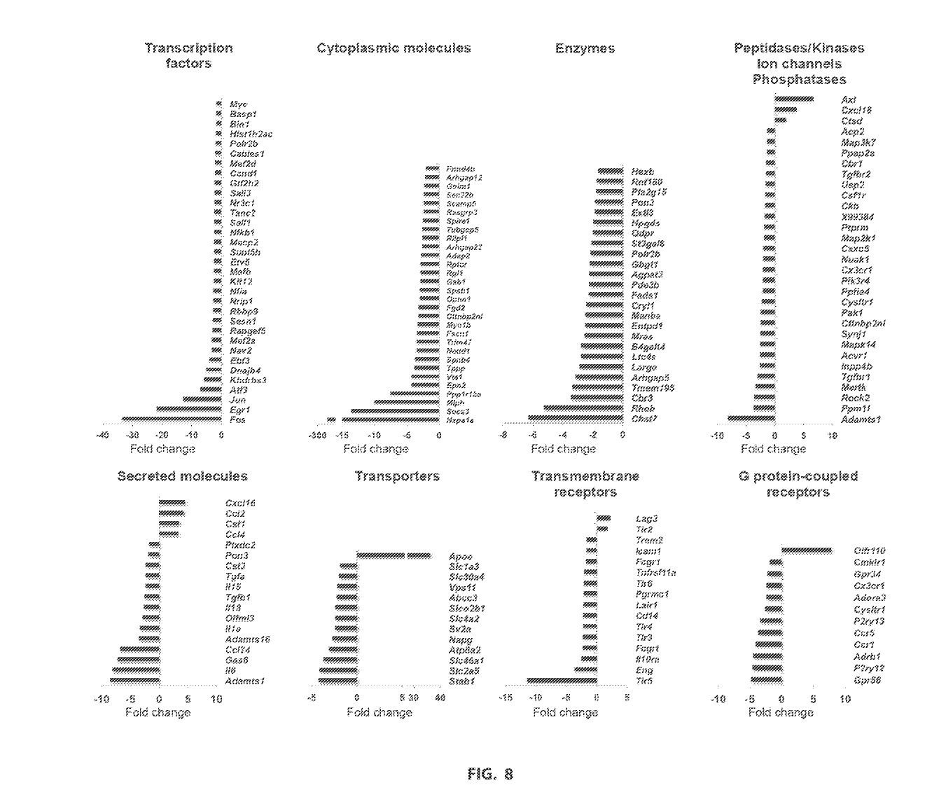

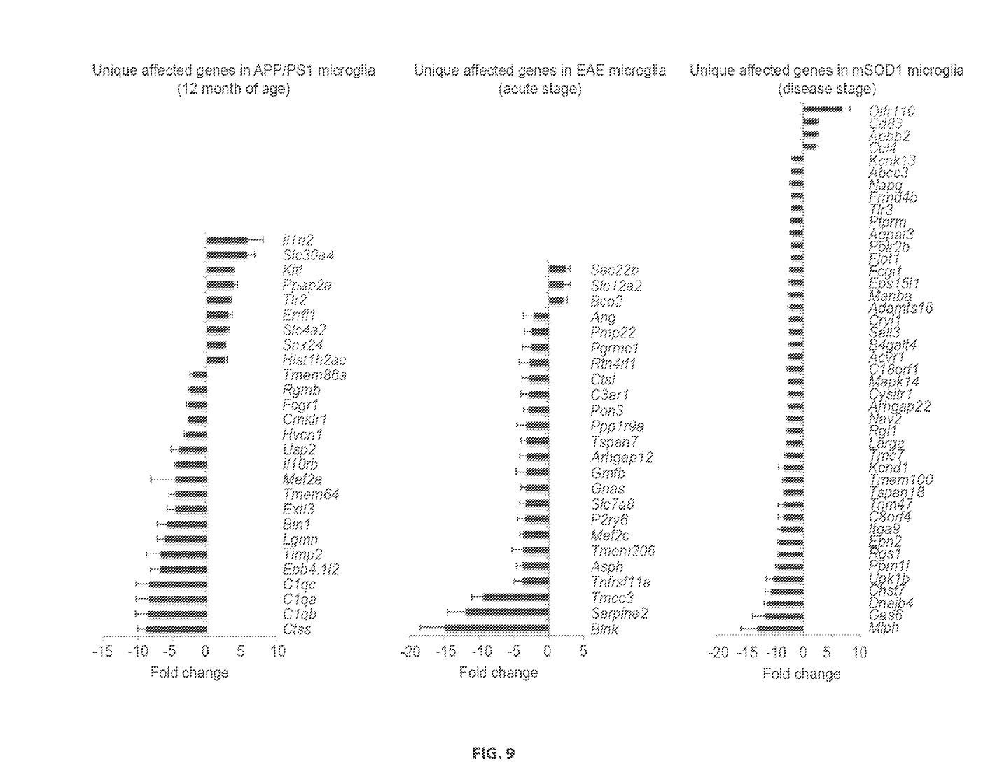

Venn diagram summarizes the number of dysregulated genes in all three diseases (FIG. 1a). Although individual differences in the expression profile in disease-associated microglia could be determined in the three investigated diseases (FIG. 9), we could identify a subset of fifty common genes that were dysregulated in all three (FIG. 1a). This universal group was characterized by severe suppression of key microglia homeostatic signature genes including P2ry12, Tgfbr1, Tmem119, Gpr34, Jun, Olfm13, Csf1r, Hexb, Mertk and Tgfb1 (FIG. 1b). We confirmed down regulation of the homeostatic signature gene P2ry12 on the protein level using confocal microscopy of P2ry12 staining on spinal cord section in different disease stages in EAE. Down regulation of P2ry12 was most obvious in onset and peak EAE. In the latter, P2ry12 was almost completely lost. Remarkably, P2ry12-positive microglia reemerges in the recovery phase (FIG. 1C).

Common upregulated genes in disease-associated microglia were Axl, Csf1 and Ccl2. Surprisingly, the most upregulated gene commonly found in disease-associated microglia was Apoe (FIG. 1b).

Expression down regulation of homeostatic signature key genes (FIG. 1c) and upregulation of proinflammatory genes (FIG. 1d) in disease-associated microglia from brain and spinal cord was abundant in different disease stages in EAE. We found remarkable upregulation of Apoe expression in acute and chronic disease stages in EAE (FIG. 1c). To rule out model specific changes, qPCR analysis of Apoe expression was performed in the EAE-NOD- and in the EAE/C57/Bl6-model, too (FIG. 10). Dramatic upregulation of Apoe expression confirmed the universal nature of Apoe as a common marker for diseased microglia. Of note, Apoe was highest in the chronic phase in the EAE-NOD-model. This is in line with our recent finding in SOD1 mice and human ALS patients: here, Apoe was upregulated in microglia from SOD1 mice and ALS subjects, and was inversely associated with the expression of the homeostatic signature in microglia (8).

Ingenuity pathway analysis (IPA) for transcriptional regulation in disease-associated microglia highlights the central role of Tgfb1 and APOE as a common signaling platform in the switch from healthy to diseased microglia (FIG. 1e). Further molecules might be centrally involved in the balance of this axis including Csf1, Jun and Axl.

### Example 2. Apoptotic Neurons Specifically Induce Apoe Expression and Leads to Suppression of Homeostatic Signature in Phagocytic Microglia

Our finding that loss of homeostatic microglia signature in combination with highly upregulated Apoe expression is a universal marker of dysregulated disease associated microglia led us to investigate potential trigger for this specific molecular pattern. Surprisingly, treatment of microglia in vitro and in vivo with LPS, a classical M1 inducing reagent, did not lead to the upregulation of Apoe expression in either (FIG. 11A-B). In contrast, LPS down regulates Apoe expression in vitro and in vivo.

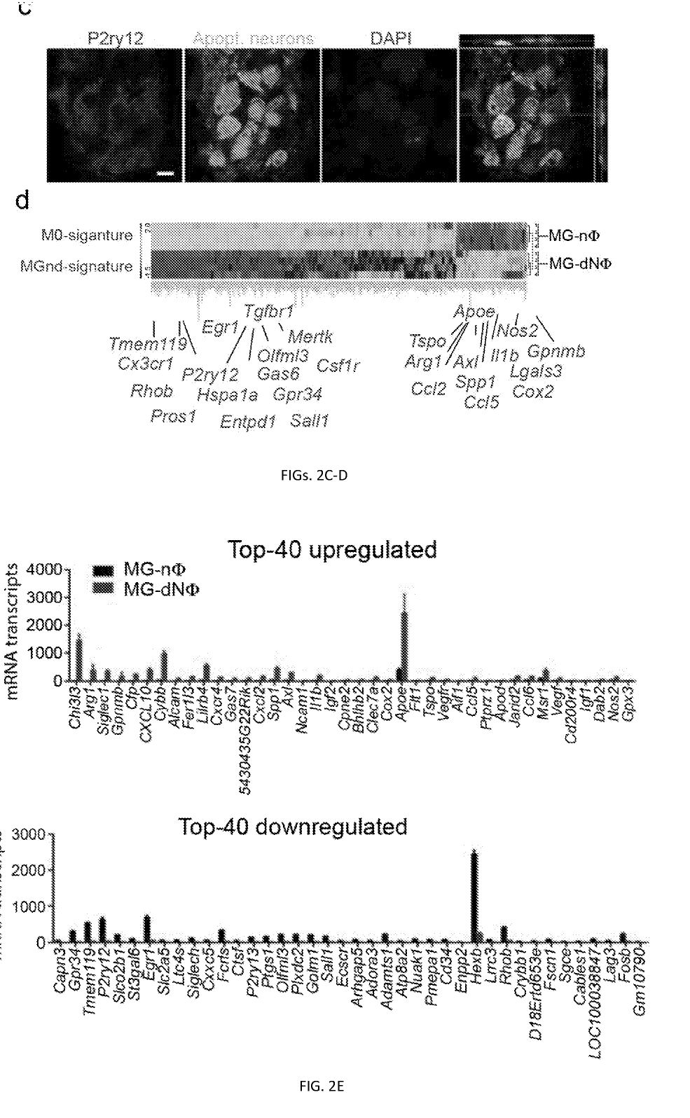

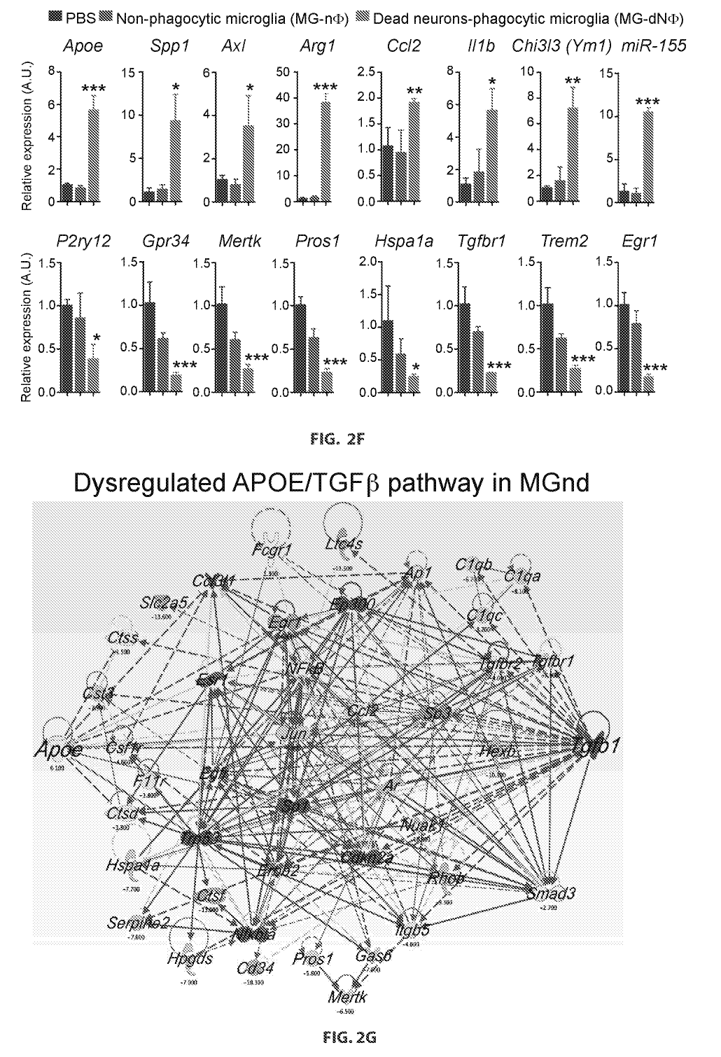

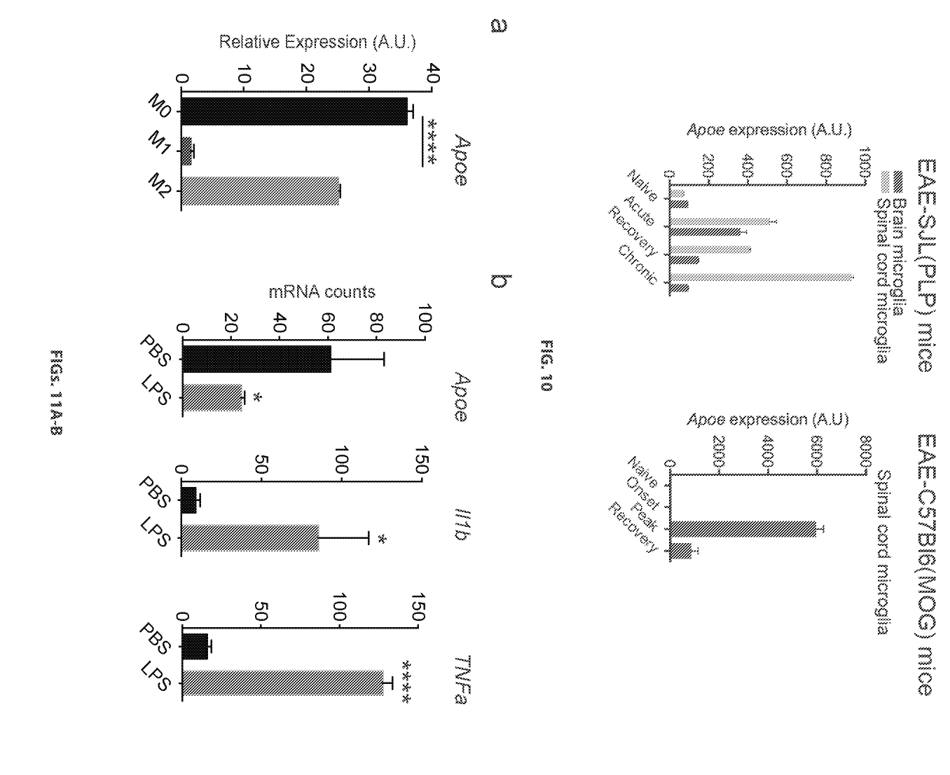

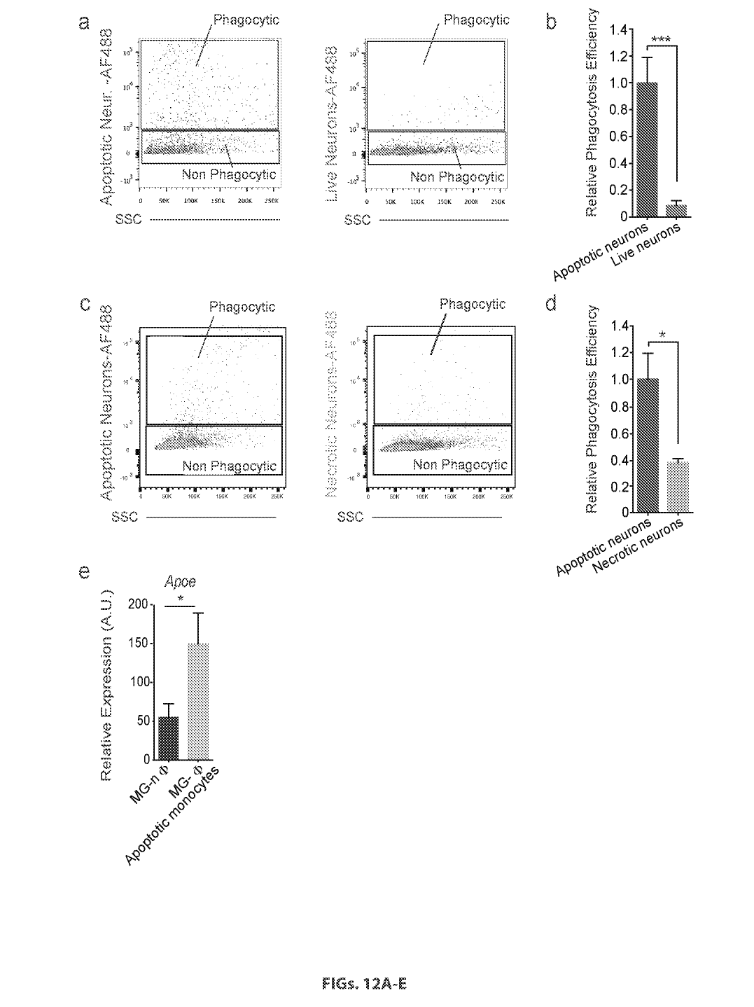

A very simple common denominator for the investigated diseases, where we identified upregulation of APOE expression in microglia, is the occurrence of neurodegeneration, namely, the degeneration and apoptosis of neurons. To investigate the impact of phagocytosis of apoptotic neurons on microglia molecular signature, we induced apoptosis in neurons with UV-light and fluorescently labeled them. Labeled neurons were stereotactically injected into cortex and hippocampus of wild type mice. PBS injection served as a control. 16h later, we isolated microglia by FACS sorting (CD11b+, FCRLS+; the latter antibody was generated in our previous study(7) and is specific for brain resident microglia) and subsequently sorted phagocytic microglia containing fluorescently labeled neurons (FIG. 2a). We found that apoptotic neurons were efficiently phagocytosed by microglia (FIG. 2a). Of note, this was in dramatic contrast to live neurons, which were not phagocytosed by healthy microglia (FIG. 12B). The efficiency to phagocytose necrotic neurons was also decreased compared to apoptotic neurons (FIG. 12A)

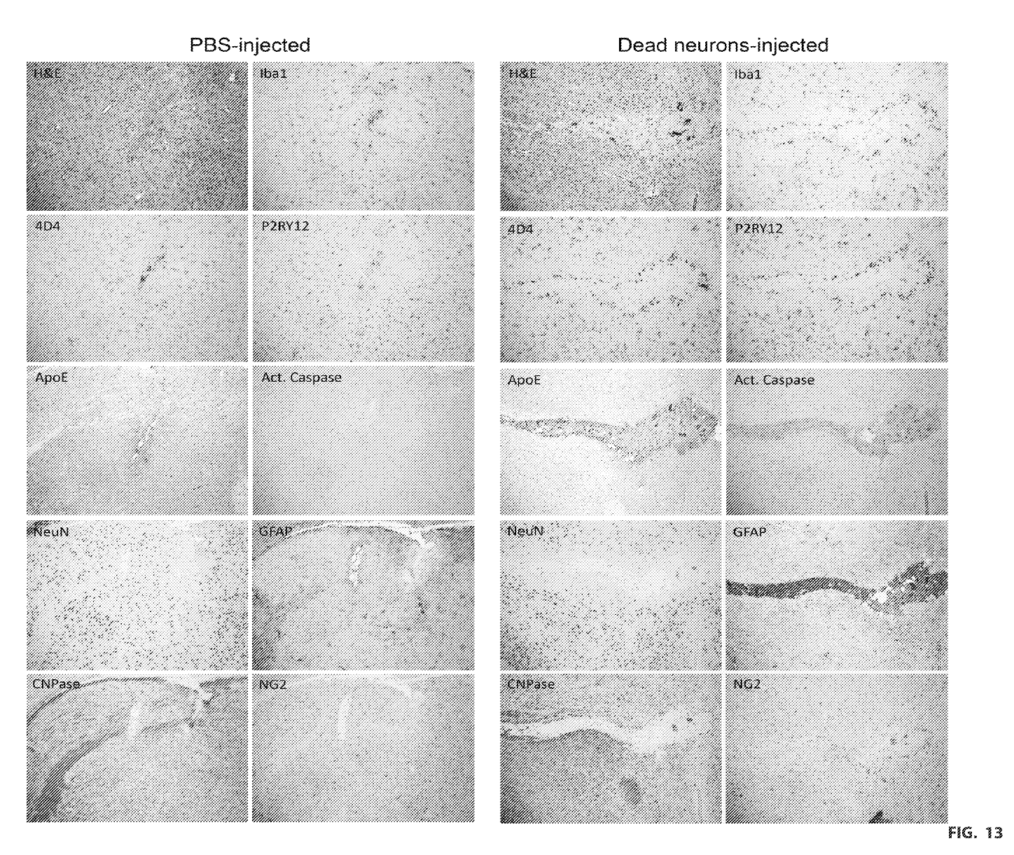

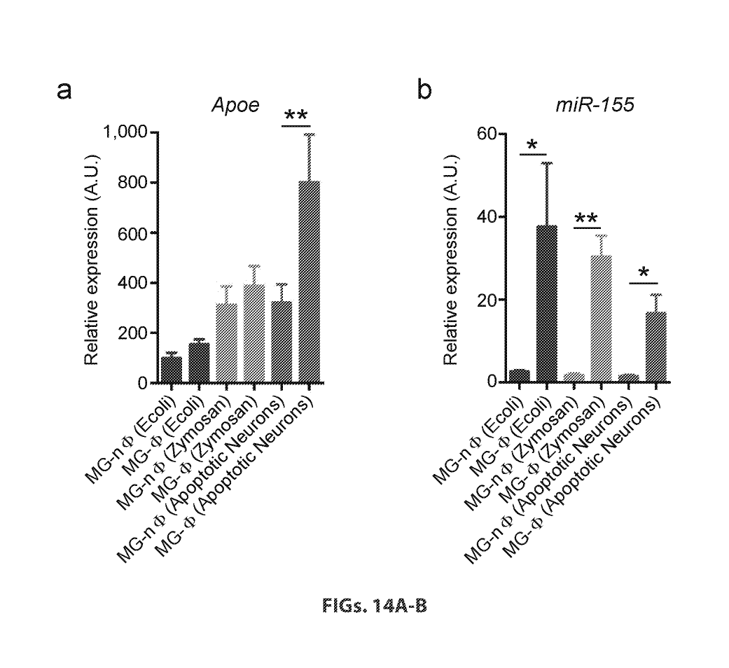

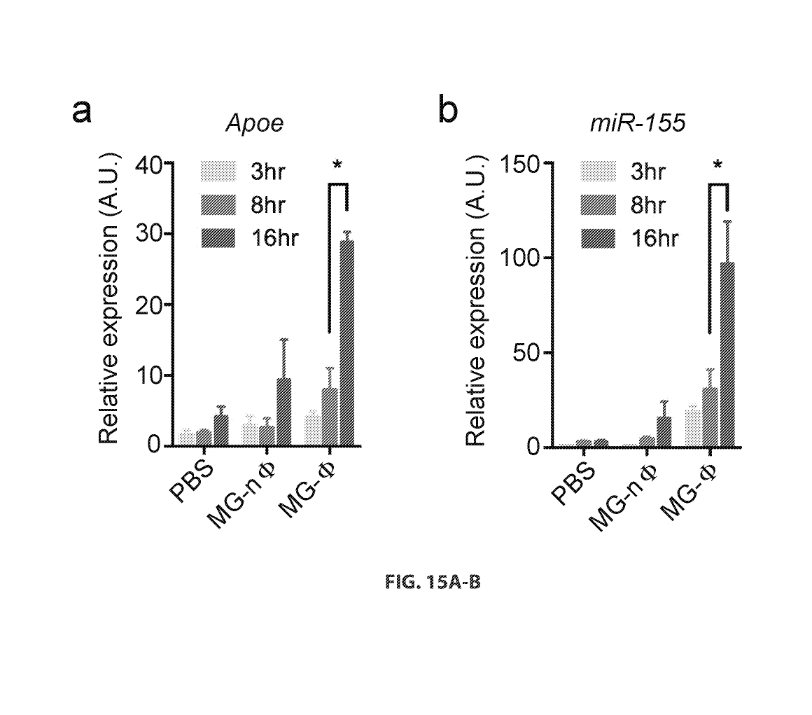

Monitoring the apoptotic neuron injection site in the brain 16 h post injection by IHC with Iba1 for microglia/monocytes and P2ry12 detecting only brain resident microglia(7), we found recruitment of microglia to the side of neuron injection, whereas injection of PBS alone did only marginally attract microglia (FIG. 2b). Moreover, injection of apoptotic neurons was accompanied with neuronal loss surrounding the injection side (FIG. 13). Using confocal microscopy, we could confirm the recruitment of microglia to the side of injection. Z-stack images showed that microglia phagocytosed entire apoptotic neurons as well as neuronal debris (FIG. 2c). To investigate whether Apoe is upregulated in phagocytic microglia, we FACS-sorted microglia from the brain 3, 8, and 16 h post injection of neurons. Using qPCR analysis, we could show that expression of Apoe and miR-155 were both significantly increase after 16 h in phagocytic microglia only (FIG. 15A-B and b/time course experiment). Therefore, we performed subsequent analyses 16 h post injection. Importantly, the upregulation of Apoe expression was specific for the phagocytosis of apoptotic neurons, whereas phagocytosis of E. coli or Zymosan does not induce Apoe in microglia (FIGS. 14A-B). In contrast, miR155 was likewise induced by phagocytosis of apoptotic neurons, E coli and Zymosan particles, suggesting that there are two independent mechanisms that induce Apoe and miR-155 in microglia (FIGS. 14A-B).

We extensively profiled the molecular signature of apoptotic-neuron-phagocytic-(MG-ancD) in comparison to non-phagocytic microglia (MG-NCD) sorted from the same brain with Nanostring gene analysis using our custom MG468 chip (including unique microglial as well as inflammatory genes (8). We found that phagocytosis of apoptotic neurons induce a microglia phenotype identical to what we identified in disease-associated microglia: homeostasis genes were dramatically suppressed in phagocytic microglia including Tmem119, Mertk, Gpr34, P2ry12, TGFbR1 and others (see expression heat map FIG. 2d), whereas Apoe was significantly upregulated. Of note, also arginase 1 (Arg1) was highly upregulated in phagocytic microglia. Arg1 is a classical marker for alternatively activated M2-microglia. On the basis of our findings, we speculated that arginase 1 is only upregulated upon phagocytosis and thus, microglia that pre-upregulated Arg1 might not exist in the brain.

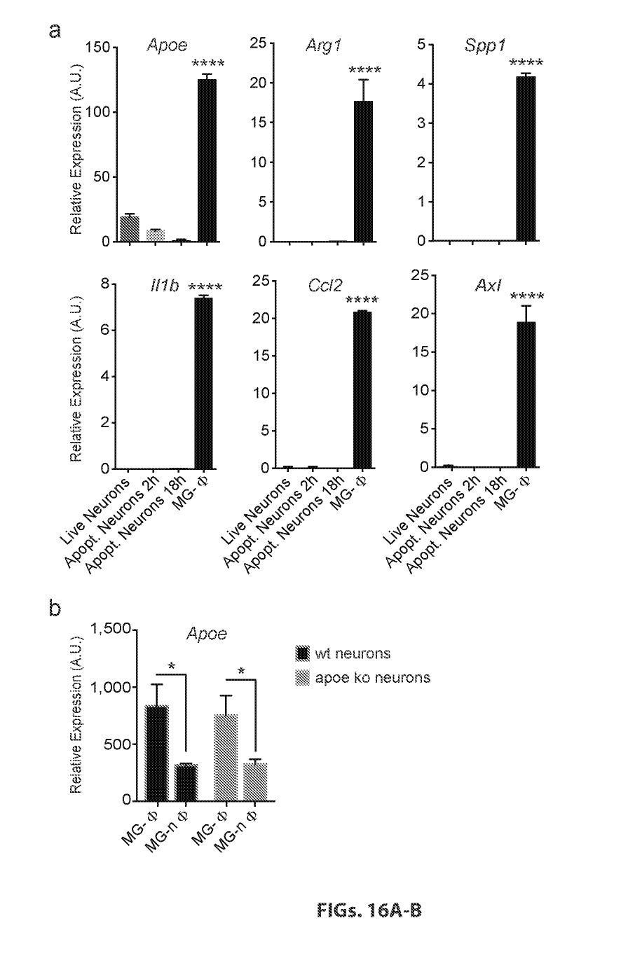

The Top-40 upregulated and downregulated genes determined by Nanostring chip analysis in phagocytic microglia are summarized in FIG. 2e. Of note, Apoe was one of the most upregulated genes. Using qPCR analysis we confirmed the suppression of selected microglial homeostasis genes like P2ry12, Gpr34, Mertk and others in phagocytic microglia (FIG. 2f). Moreover, upregulation of Apoe, Spp1, Axl, Arg1 and a variety of proinflammatory genes including Ccl2, Il1b, miR-155 in phagocytic microglia could also be confirmed. To rule out the contribution of neuronal RNAs from phagocytosed apoptotic neurons to the detected microglia phenotype, we profiled different neuronal preparations in comparison to phagocytic microglia by qPCR (FIG. 16a). This analysis confirmed that key genes that were upregulated in phagocytic microglia were not at all or only mildly expressed in these neurons (FIG. 16a). Moreover, we used apoptotic neurons from Apoe-KO mice in our phagocytosis assay. Here, we likewise saw upregulation of Apoe expression in phagocytic microglia, confirming that neuronal RNA was not contributing to the Apoe-expression pattern of phagocytic microglia (FIG. 16b).

Ingenuity pathway analysis (IPA) visualizes transcription regulation and connections of the APOE-TGFb1 signaling axis in phagocytic microglia (FIG. 2g). IPA based on the MG468 profile showed that dysregulation of the APOE-TGFb1 signaling axis comprises several genes that were highly specific for microglia (FIG. 2g). Most microglial biological functions were suppressed in apoptotic neuron phagocytic microglia.

### Example 3. Suppression of the Homeostatic Molecular Signature in Phagocytic Microglia is Mediated by APOE Pathway but not miR-155

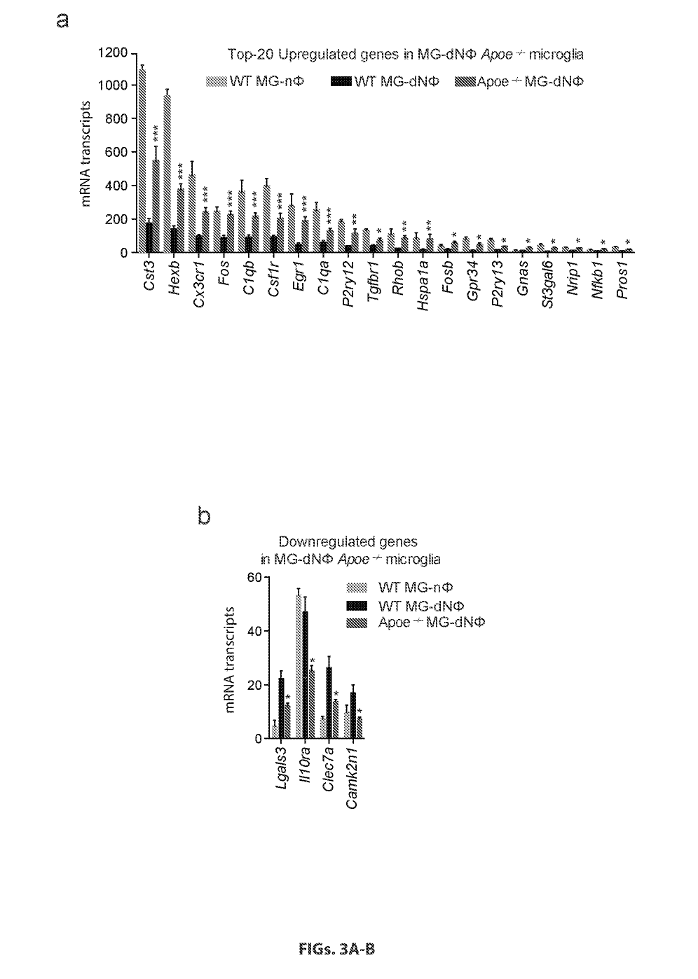

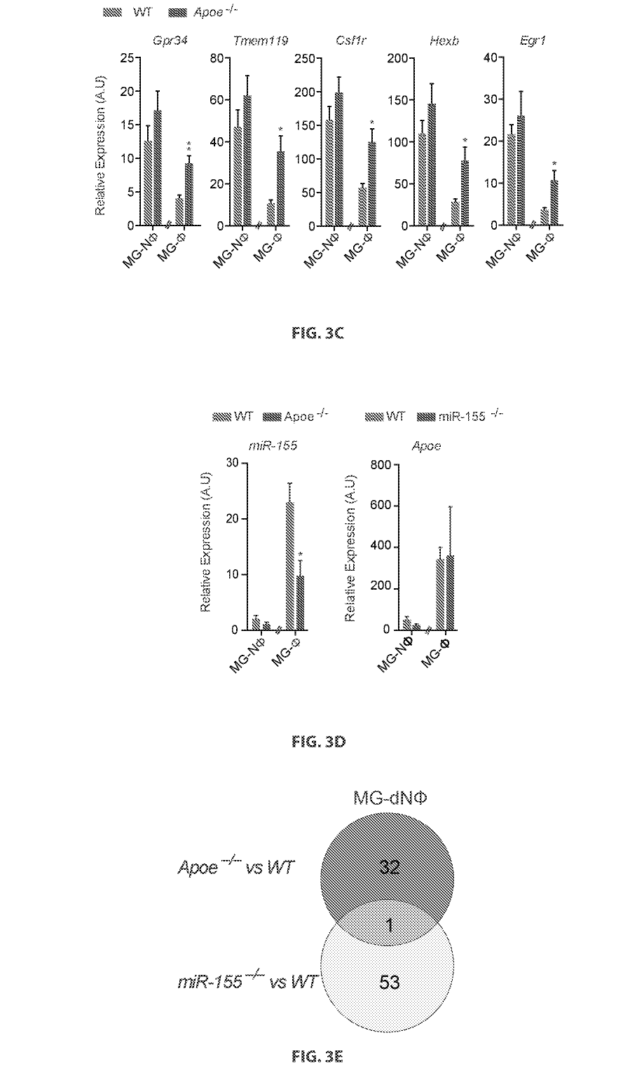

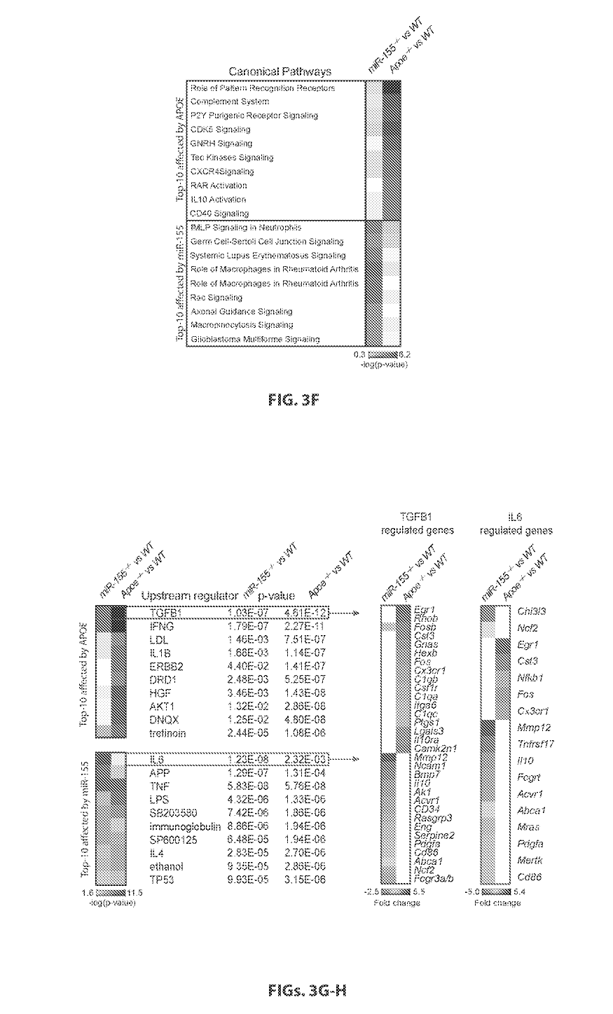

To determine the implication of APOE in the switch of molecular signature in disease-associated microglia phenotype, we stereotactically injected apoptotic neurons in APOE^{-/-} vs. WT mice. 16 h after the injection, in comparison to microglia isolated from WT mice, gene expression of markers representing microglia homeostatic signature including P2ry12, Fcrls, Tmem119, Csf1r, Cx3cr1, Hexb and Egr1 was reversed in phagocytic Apoe^{-/-} microglia (FIG. 3 a;b;d). The signature was not affected in non-phagocytic microglia from both genotypes (FIG. 3 a). We and others previously showed that miR-155 expression is upregulated in both SOD1 mice (8, 9) (10) and sporadic and familial ALS patients (8) associated with dysregulation of microglia homeostatic signature and upregulation of Apoe expression. 16 h after apoptotic neurons stereotactic injection, miR-155 expression was upregulated in WT phagocytic microglia but less in Apoe^{-/-} microglia (FIG. 3 e) consistent with miR-155 implicated in APOE pathway in phagocytic microglia. In contrast, Apoe expression upon phagocytosis of apoptotic neurons was not affected in miR-155^{-/-} mice (FIG. 3 f). Thus, miR-155 induction by phagocytosis of apoptotic neurons was regulated by APOE pathway. Interestingly, 16 h after stereotactic injection of apoptotic neurons in miR-155^{-/-} mice, we did not observe changes in gene expression of homeostatic microglia signature compared to WT microglia. Thus, APOE is at the crossroad of different microglia pathways (phagocytosis, inflammatory secretion, recruitment of peripheral cells).

### Example 4. Mertk Via Egr1 Suppresses APOE Pathway in Homeostatic Microglia

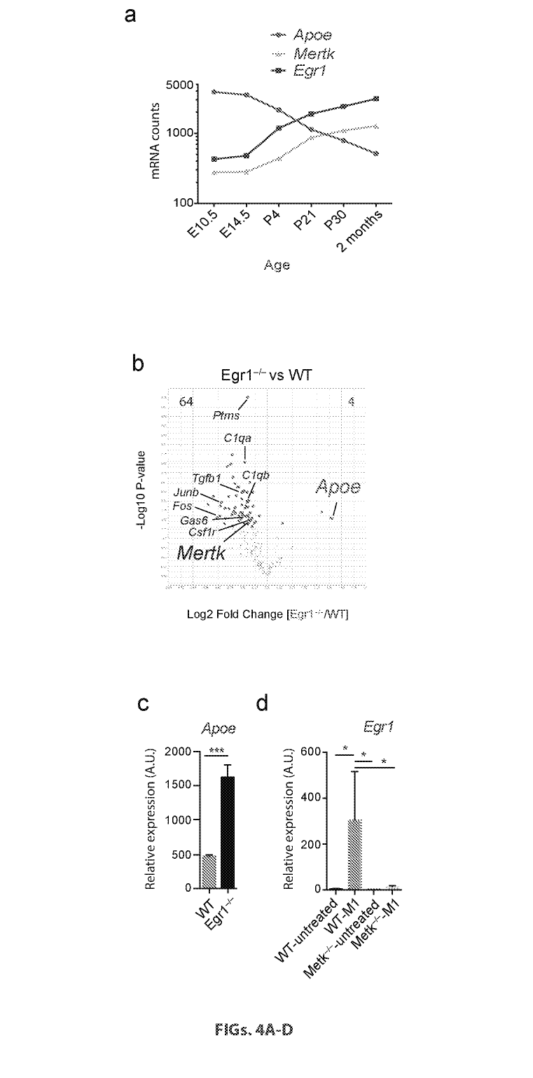

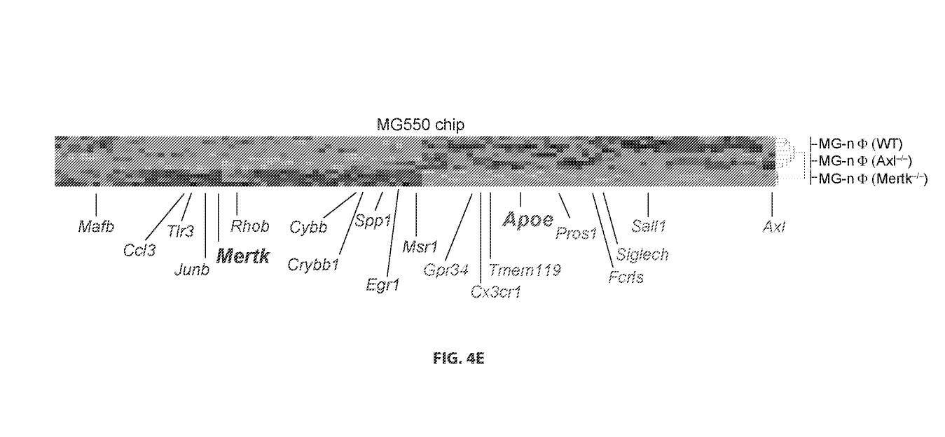

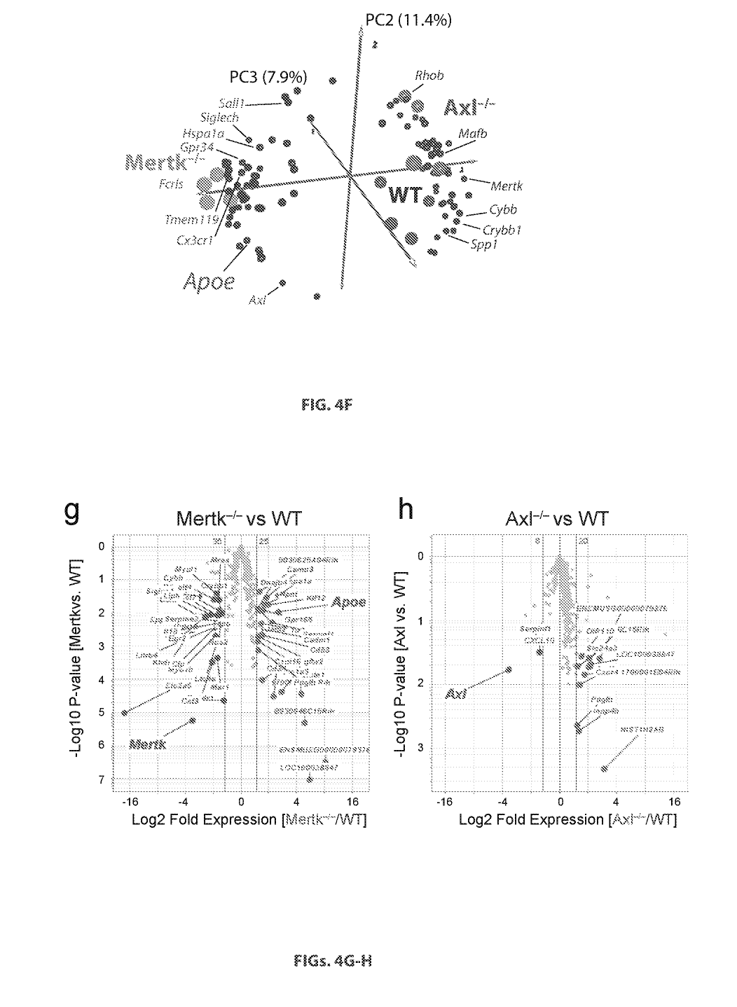

We found that microglia during disease progression in mouse models of EAE, SOD1 and APP/PS1 show increased expression of APOE pathway including Axl and inflammation-related molecules which was inversely correlated with suppression of homeostasis genes like Mertk (FIG. 1b). Similarly, the expression of these pathways was induced by apoptotic neurons (FIGS. 2d and 2f). Interestingly, we found high expression of APOE in microglia during development which was correlated with massive cell apoptosis. The APOE expression was reciprocally correlated with Mertk and EGR1 (FIG. 4a). This we hypothesized that EGR1 and MERTK are suppressors of APOE pathway in homeostatic microiglia. In order to address this question, we profiled FACS-sorted microglia from brains of WT vs EGR1−/− mice with MG550 Nanostring chip. We wound that APOE was major upregulated gene in EGR1−/− microglia (FIG. 4b, c) and both APOE and EGR1 were reciprocally expressed in Mertk−/− microglia (FIG. 4e-i). Both Mertk and Axl are receptors of TAM family of receptor tyrosine kinases and have been implicated in the phagocytosis of apoptotic neurons (11).

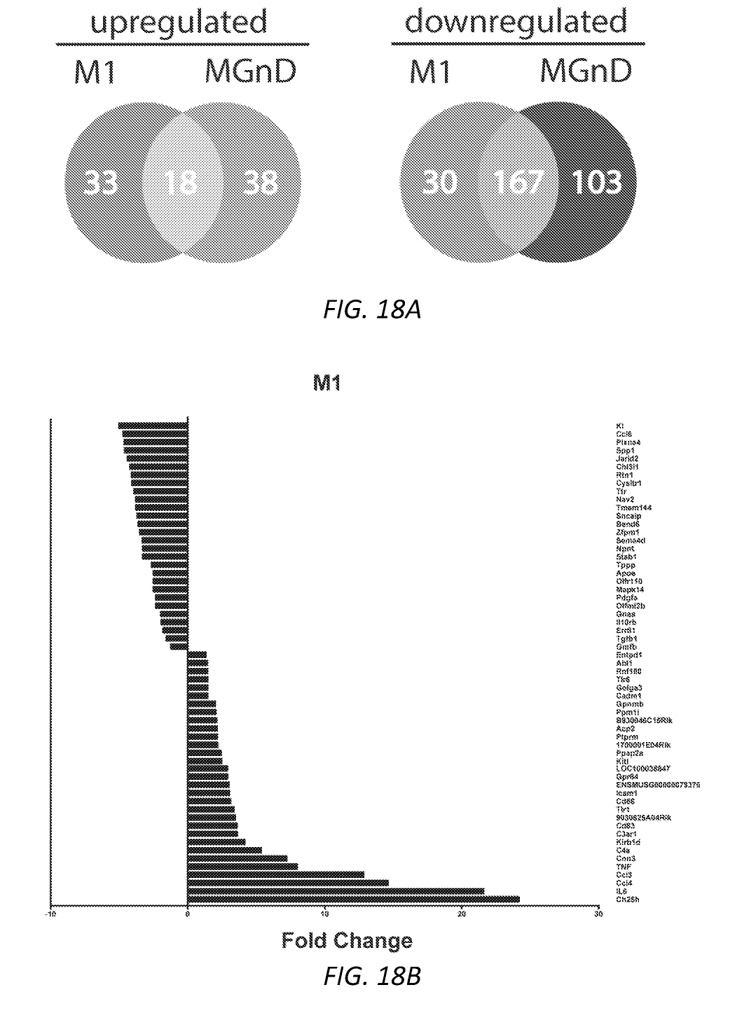

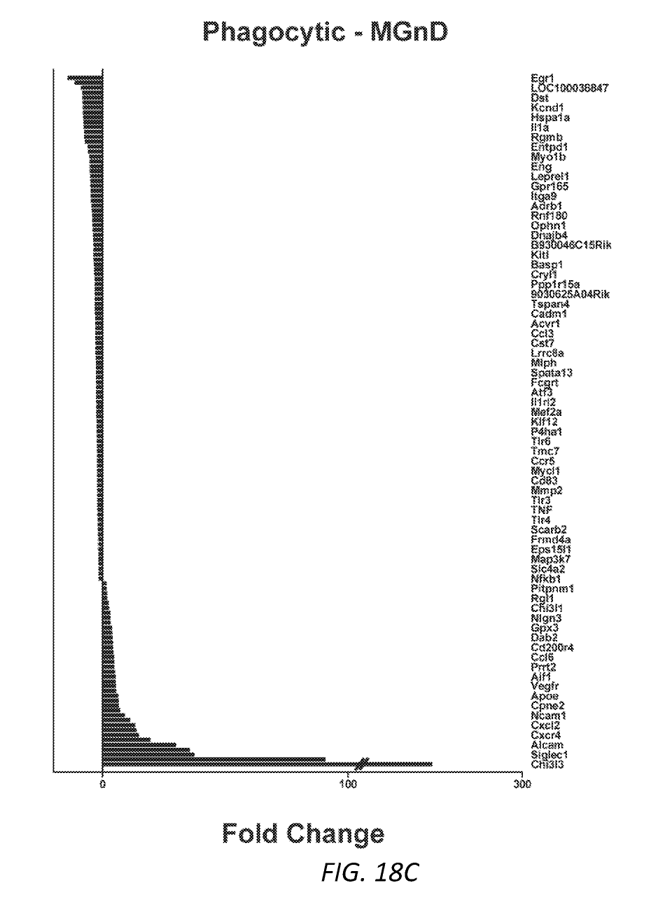

In addition, we found that a classical induced M1 inflammatory microglia phenotype induced by LPS/IFNγ does not represent a phenotype associated with neurodegenerative diseases. APOE expression was suppressed in M1 microglia (FIG. 11A-B) as an opposite to MGnd microglia phenotype induced in AD, ALS and MS mouse models (FIG. 1) or by apoptotic neurons (FIG. 2). Importantly, in addition to APOE as being a unique to MGnd vs M1 microglia, we identified a unique gene signature encompassing 141 genes in MGnd including EGR1 as the most suppressed gene (FIG. 18A-C).

### Example 5. Apoptotic Neurons Initiate APOE Pathway Via Phosphatidylserine Recognition and Trem2 Signaling

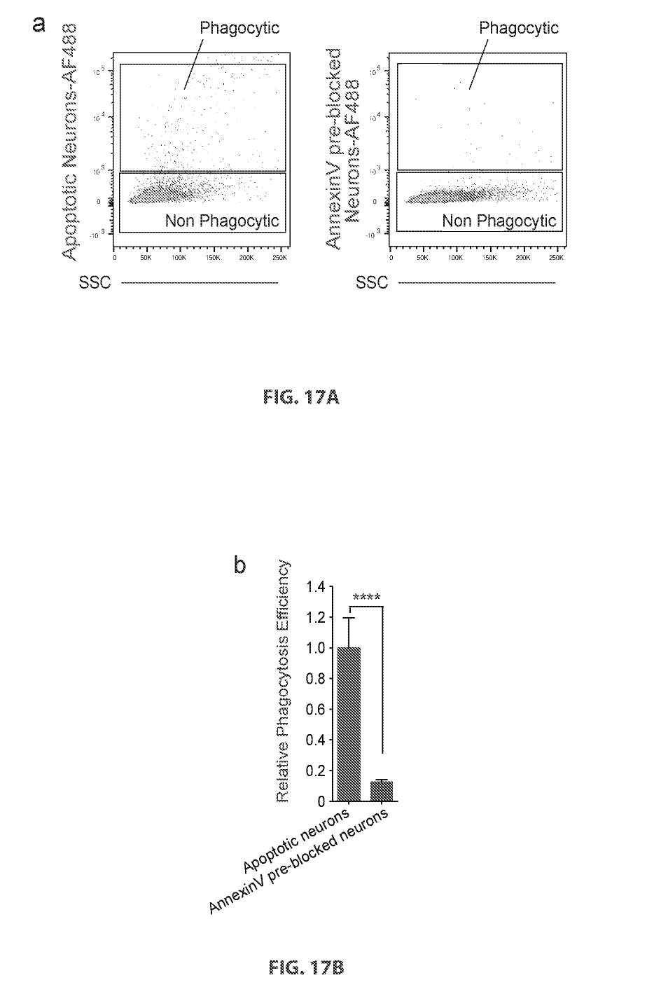

To identify specific players that initiate the APOE-pathway, we next asked whether upregulation of Apoe was exclusive for the phagocytosis of apoptotic cells. Apoptotic neurons were labeled with AnnexinV and 7AAD and FACS-sorted before stereotaxic injection. Beside cell shrinkage and DNA-fragmentation, the translocation of phosphatidylserine to the outer leaflet of the plasma membrane is a hallmark of apoptosis. AnnexinV has been shown to specifically bind to and block phosphatidylserine. To our surprise, blocking of phosphatidylserine by AnnexinV reduced the phagocytosis of apoptotic neurons by almost 90%. Moreover, our result show that exposure of phosphatidylserine on the neuronal membrane is a prerequisite for phagocytosis of apoptotic cells by microglia (FIG. 17A-B).

Recently, it has been shown that Trem2 is a sensing molecule for damage-associated lipid-patterns in neurodegeneration that might be exposed on the surface of damaged neurons (12). Thus, we hypothesized that dead neurons engage TREM2 via exposed lipids i.e. phosphatidylserine and activate the downstream APOE pathway.

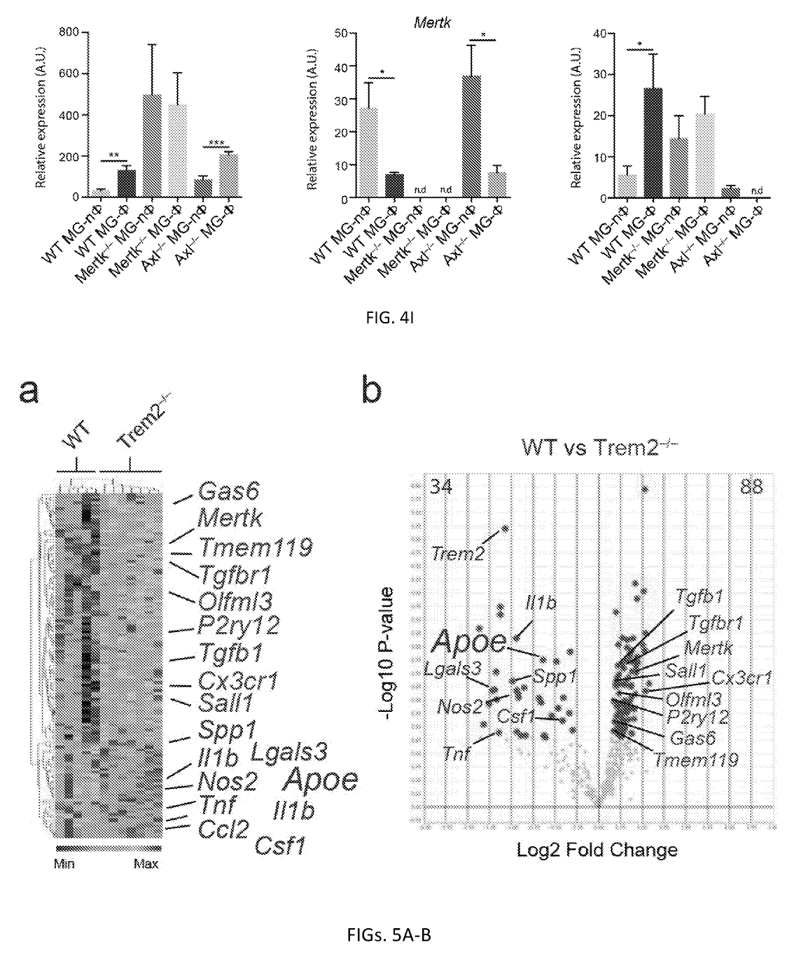

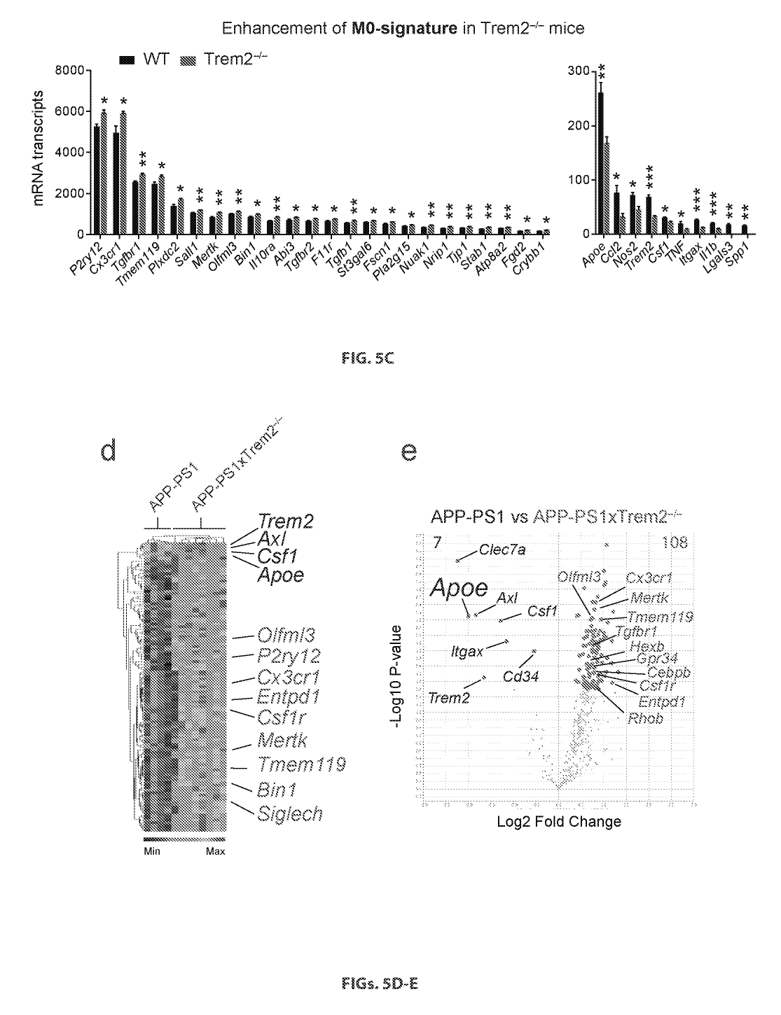

In order to address this question, we isolated FCRLS+ microglia from brain of WT and TREM2−/− mice. We found that genetic ablation of TREM2 enhances the M0-homeostatic molecular signature. Importantly, APOE was the most downregulated genes in TREM2−/− microglia (FIG. 5a-c). Most importantly, genetic ablation of TREM2 in APP/PS1 mice, a mouse model of AD, restored MGnd (microglia neurodegenerative phenotype, see FIGS. 1-3) to homeostatic microglia (FIG. 5d-f). These results confirmed that TREM2 via APOE pathway induces a neurodegenerative microglia.
1. Prinz M, Tay T L, Wolf Y, Jung S. 2014. Microglia: unique and common features with other tissue macrophages. Acta Neuropathol
2. Tremblay M E, Stevens B, Sierra A, Wake H, Bessis A, Nimmerjahn A. 2011. The role of microglia in the healthy brain. J Neurosci 31: 16064-9
3. Prinz M, Priller J, Sisodia S S, Ransohoff R M. 2011. Heterogeneity of CNS myeloid cells and their roles in neurodegeneration. Nat Neurosci 14: 1227-35
4. Sierra A, Encinas J M, Deudero J J, Chancey J H, Enikolopov G, Overstreet-Wadiche L S, Tsirka S E, Maletic-Savatic M. 2010. Microglia shape adult hippocampal neurogenesis through apoptosis-coupled phagocytosis. Cell Stem Cell 7: 483-95
5. Guerreiro R, Wojtas A, Bras J, Carrasquillo M, Rogaeva E, Majounie E, Cruchaga C, Sassi C, Kauwe J S, Younkin S, Hazrati L, Collinge J, Pocock J, Lashley T, Williams J, Lambert J C, Amouyel P, Goate A, Rademakers R, Morgan K, Powell J, St George-Hyslop P, Singleton A, Hardy J, Alzheimer Genetic Analysis G. 2013. TREM2 variants in Alzheimer's disease. New England Journal of Medicine 368: 117-27
6. Kleinberger G, Yamanishi Y, Suarez-Calvet M, Czirr E, Lohmann E, Cuyvers E, Struyfs H, Pettkus N, Wenninger-Weinzierl A, Mazaheri F, Tahirovic S, Lleó A, Alcolea D, Fortea J, Willem M, Lammich S, Molinuevo J L, Sanchez-Valle R, Antonell A, Ramirez A, Heneka M T, Sleegers K, van der Zee J, Martin J-J, Engelborghs S, Demirtas-Tatlidede A, Zetterberg H, Van Broeckhoven C, Gurvit H, Wyss-Coray T, Hardy J, Colonna M, Haass C. 2014. TREM2 mutations implicated in neurodegeneration impair cell surface transport and phagocytosis. Science Translational Medicine 6: 243ra86
7. Butovsky O, Jedrychowski M P, Moore C S, Cialic R, Lanser A J, Gabriely G, Koeglsperger T, Dake B, Wu P M, Doykan C E, Fanek Z, Liu L, Chen Z, Rothstein J D, Ransohoff R M, Gygi S P, Antel J P, Weiner H L. 2014. Identification of a unique TGF-beta-dependent molecular and functional signature in microglia. Nat Neurosci 17: 131-43
8. Butovsky O, Jedrychowski M P, Cialic R, Krasemann S, Murugaiyan G, Fanek Z, Greco D J, Wu P M, Doykan C E, Kiner O, Lawson R J, Frosch M P, Pochet N, Fatimy R E, Krichevsky A M, Gygi S P, Lassmann H, Berry J, Cudkowicz M E, Weiner H L. 2015. Targeting miR-155 restores abnormal microglia and attenuates disease in SOD1 mice. Ann Neurol 77: 75-99
9. Butovsky O, Siddiqui S, Gabriely G, Lanser A J, Dake B, Murugaiyan G, Doykan C E, Wu P M, Gali R R, Iyer L K, Lawson R, Berry J, Krichevsky A M, Cudkowicz M E, Weiner H L. 2012. Modulating inflammatory monocytes with a unique microRNA gene signature ameliorates murine ALS. J Clin Invest 122: 3063-87
10. Koval E D, Shaner C, Zhang P, du Maine X, Fischer K, Tay J, Chau B N, Wu G F, Miller T M. 2013. Method for widespread microRNA-155 inhibition prolongs survival in ALS-model mice. Hum Mol Genet
11. Zagorska A, Traves P G, Lew E D, Dransfield I, Lemke G. 2014. Diversification of TAM receptor tyrosine kinase function. Nat Immunol 15: 920-8

12. Wang Y, Cella M, Mallinson K, Ulrich J D, Young K L, Robinette M L, Gilfillan S, Krishnan G M, Sudhakar S, Zinselmeyer B H, Holtzman D M, Cirrito J R, Colonna M. 2015. TREM2 lipid sensing sustains the microglial response in an Alzheimer's disease model. Cell 160: 1061-71

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating amyotrophic lateral sclerosis (ALS) in a subject, the method comprising administering to a subject having ALS a therapeutically effective amount of at least one inhibitor of Trem2 or ApoE.

2. The method of claim 1, wherein the at least one inhibitor of Trem2 and/or ApoE is an inhibitory nucleic acids targeting Trem2 and/or ApoE, or an antibody that binds to and inhibit Trem2 or ApoE.

3. (canceled)

4. (canceled)

5. A method of treating amyotrophic lateral sclerosis (ALS) in a subject, the method comprising administering to a subject having ALS a therapeutically effective amount of at least one sense nucleic acid encoding Egr1 and/or Mertk.

6. The method of claim 2, wherein the at least one inhibitory nucleic acid is an antisense oligonucleotide or small interfering RNA.

7. The method of claim 5, wherein the nucleic acid is in a viral vector.

8. The method of claim 2, wherein the nucleic acid or antibody is injected into the cerebrospinal fluid of a subject.

9. The method of claim 2, wherein the nucleic acid or antibody is administered by intracranial injection or intrathecal injection.

10. The method of claim 2, wherein the at least one inhibitory nucleic acid is complexed with one or more cationic polymers and/or cationic lipids.

11. The method of claim 5, wherein the nucleic acid is injected into the cerebrospinal fluid of a subject.

12. The method of claim 5, wherein the nucleic acid is administered by intracranial injection or intrathecal injection.
